(19) United States
(12) Patent Application Publication
(10) Pub. No.: US 2024/0075152 A1
(43) Pub. Date: Mar. 7, 2024

(54) SUPRAMOLECULAR ASSEMBLIES, COMPOSITIONS AND METHODS FOR PRODUCING AND USING THE SAME

(71) Applicant: PURDUE RESEARCH FOUNDATION, West Lafayette, IN (US)

(72) Inventors: Jean CHMIELEWSKI, Lafayette, IN (US); Ryan CURTIS, Indianapolis, IN (US); Michael David JORGENSEN, Hoffman Estates, IL (US)

(21) Appl. No.: 18/261,264

(22) PCT Filed: Jan. 13, 2022
(86) PCT No.: PCT/US2022/012360

§ 371 Date: Jul. 12, 2023

Related U.S. Application Data
(60) Provisional application No. 63/136,762, filed on Jan. 13, 2021.

(51) Int. Cl. A61K 47/64; A61K 47/52; C07K 14/395

(52) U.S. Cl. CPC A61K 47/64; A61K 47/52; C07K 14/395; C07K 2299/00; C07K 2319/21; C07K 2319/73

(57) ABSTRACT

Supramolecular assemblies, compositions thereof, and methods for preparing and use the same are provided. The supramolecular assemblies comprise a trimeric variant of a GCN4 peptide capable of self-assembly and incorporating one or more guest molecules in a functional 3D native structure.

### PRIORITY

This patent application is related to and claims the priority benefit of U.S. Provisional Patent Application No. 63/136,762 filed Jan. 13, 2021, the content of which is hereby incorporated by reference in their entirety into this disclosure.

### STATEMENT REGARDING FEDERALLY SPONSORED RESEARCH

This invention was made with U.S. government support under 2108722-CHE, awarded by the National Science Foundation. The United States Government has certain rights in the invention.

### TECHNICAL FIELD

This disclosure generally relates supramolecular assemblies comprising crystalline biomaterial arrays and their modes of syntheses. Further, methods for isolating functional proteins reversibly within such supramolecular assemblies are provided.

### SEQUENCE LISTINGS

The sequences herein (SEQ ID NOS: 1-6) are also provided in computer readable form encoded in a file filed herewith and incorporated herein by reference. The information recorded in computer readable form is identical to the written Sequence Listings provided below, pursuant to 37 C.F.R. § 1.821(f).

### BACKGROUND

A challenge in the field of bio-nanotechnology is the development of nano-sized delivery systems capable of carrying bioactive substances to a predefined site and unloading them in a controlled manner. Further, when delivered, the bioactive substances contained within the delivery system (i.e. the “cargo”) need to retain their function for the system to be effective. This depends greatly on the ability to incorporate cargoes having a diverse range of functionalities into these biomaterials with precise spatial control, which has historically proven difficult to achieve.

Coiled coil peptides have been used as building blocks to generate a variety of higher order assemblies, including fibers, nanoblocks, spherical cages, nanotubes, crystals, hydrogels, and three dimensional (3D) matrices. In some cases, these assemblies have been loaded with cargo, such as fluorophores, dextrans, peptides, and proteins.

Incorporating cargoes with a diverse range of functionalities into such biomaterials with precise spatial control has been an important challenge in biotechnology. Proteins represent a particularly intriguing cargo as they perform a wide variety of functions; however, their complexity also makes their inclusion within biomaterials in a fully folded form (i.e. functional form) a challenge. Aggregation can be a critical issue during the storage of proteins, and at elevated temperatures protein folding followed by aggregation is a major mechanism for loss of function. Preserving the tertiary structure of proteins within biomaterials could, at a minimum, facilitate the development of robust enzyme catalysts and/or eliminate conventional “cold chain” storage and transport barriers in the development of biopharmaceuticals.

In view of the foregoing, it is an object of the present disclosure to provide materials and methods to facilitate a self-assembling and stable assemblies that can reversibly host one or more proteins therein while preserving tertiary structure and stability (e.g., thermal) of such protein cargo. The isolation of proteins reversibly within crystalline biomaterials could be a powerful means to confront the conventional challenges in the field. This and other objectives and advantages, as well as inventive features, will become apparent from the detailed description provided herein.

### SUMMARY

A supramolecular assembly is provided, such assembly comprising a first set of peptide units and at least one histidine-tagged (His-tagged) cargo, with the first set of peptide units forming a three-dimensional (3D) crystal (e.g., a hexagonal 3D crystal). In certain embodiments, each peptide unit of the first set comprises a trimeric coiled-coil peptide (e.g., encoded by SEQ ID NO: 1 or has at least 75% sequence identity or more, at least 85% sequence identity or more, at least 90% sequence identity or more, or at least 95% sequence identity or more to SEQ ID NO: 1) comprising a first metal-binding ligand fused to a first end of a trimeric variant of a GCN4 peptide and a second metal-binding ligand fused to a second end of the trimeric variant of a GCN4 peptide. The first end of the trimeric variant can be an N-terminus and the second end of the trimeric variant can be a C-terminus.

The at least one his-tagged cargo can be reversibly incorporated into the 3D crystal. Notably, each cargo incorporated (or loaded) in the 3D crystal retains a functional 3D native structure and is independently organized within or on the 3D crystal. The at least one cargo can comprise a His-tagged, fully folded protein. In certain embodiments, the at least one cargo is a His-tagged fluorescent molecule. In certain embodiments, the at least one cargo is a His-tagged oligonucleotide. In certain embodiments, the at least one cargo is a His-tagged therapeutic agent. In certain embodiments, the at least one cargo is a His-tagged pharmaceutical compound or a His-tagged pharmaceutically acceptable salt thereof. Further, the at least one cargo can be a His-tagged enhanced green fluorescent protein with an N-terminal His₆-tag (EGFP). There, the EGFP can be incorporated into the 3D crystal in an ordered, hourglass pattern. Additionally or alternatively, the at least one cargo reversibly incorporated into the 3D crystal can comprise a protein and, when the assembly is stored at room temperature, the protein does not undergo substantial denaturation of its functional 3D native structure.

In certain exemplary embodiments, the 3D crystal undergoes facile dissolution in the presence of a chelator and releases the at least one cargo retaining its functional 3D native structure. In certain exemplary embodiments, the assembly can further comprise one or more metal ions to promote self-assembly of the 3D crystal, wherein the one or more metal ions are linked to the metal-binding ligands of each peptide unit of the first set. Such linkages can comprise, for example, coordinate covalent bonds, noncovalent bonds, or a combination thereof.

The one or more metal ions can be divalent metal ions, trivalent metal ions, or a combination of divalent and trivalent metal ions. In certain embodiments, the one or more metal ions are selected from the group consisting of Ni²⁺, Zn²⁺, Cu²⁺, Co²⁺, Fe²⁺, Co²⁺, Fe³⁺, Rh³⁺, Ru³⁺, and Gd³⁺.

The supramolecular assembly can optionally further comprise a second set of peptide units, with each peptide unit of the second set comprising a trimeric coiled-coil peptide without metal-binding ligands fused thereto. In such embodiments, the first and second sets of peptide units, in combination, form the 3D crystal and a metal source (e.g., a metal or metal ion) is not needed to promote assembly.

The supramolecular assembly of claim 18, wherein the trimeric coiled-coil peptide of the second set is encoded by SEQ ID NO: 2 or has at least 75% sequence identity or more, at least 85% sequence identity or more, at least 90% sequence identity or more, or at least 95% sequence identity or more to SEQ ID NO: 2.

Each trimeric coiled-coil peptide of the first and/or second set can have at least 75% sequence identity or more, at least 85% sequence identity or more, at least 90% sequence identity or more, or at least 95% sequence identity or more to SEQ ID NO: 1. In certain embodiments, each trimeric coiled-coil peptide of the second set is encoded by SEQ ID NO: 2. In certain embodiments, each trimeric coiled-coil peptide of the second set is encoded by SEQ ID NO: 1. In certain embodiments, SEQ ID NO: 2 further comprises a N-terminal acetylation and a C-terminal amino acid.

Now referring to the metal-binding ligands, the first metal-binding ligand can comprise nitrilotriacetic acid (NTA) and/or the second metal-binding ligand can comprise a di-histidine (His₂). In certain embodiments (metal-promoted or not), the first metal-binding ligand is NTA and the second metal-binding ligand is His₂.

Methods are also provided for the preparation of a supramolecular assembly described herein. In certain embodiments, such methods comprise combining a plurality of peptide units and a plurality of his-tagged cargo units to generate a composition, each peptide unit comprising a trimeric coiled-coil peptide comprising a first metal-binding ligand fused to a first end of a trimeric variant of a GCN4 peptide and a second metal-binding ligand fused to a second end of the trimeric variant of a GCN4 peptide and the composition comprising between about a 30:1 and about a 70:1 ratio of peptide unit to his-tagged cargo unit, wherein the composition is a 3D crystal, and the plurality of his-tagged cargo units are reversibly incorporated and independently organized into the 3D crystal, and the incorporated cargo units retain a functional 3D native structure of each cargo unit.

In some embodiments, the method can further comprise combining a metal source with the plurality of peptide units and the plurality of his-tagged cargo units to generate the composition. Further, the cargo units can comprise a protein, a fluorescent molecule, an oligonucleotide, or a combination of two or more of the foregoing. In certain exemplary embodiments, the cargo units comprise a therapeutic agent. In certain exemplary embodiments, the cargo units comprise a protein.

The metal source can be one or more metal ions (e.g., divalent or trivalent metal ions).

In certain embodiments of such methods, the plurality of peptide units comprises a first set of peptide units and a second set of peptide units, with each peptide unit of the first set comprises a trimeric coiled-coil peptide comprising a first metal-binding ligand fused to a first end of a trimeric variant of a GCN4 peptide and a second metal-binding ligand fused to a second end of the trimeric variant of a GCN4 peptide. There, each peptide unit of the second set can comprise a trimeric coiled-coil peptide without metal-binding ligands fused thereto. Such methods can be metal free.

Additional methods for treating a subject experiencing or at risk for experiencing a disease state are provided. In certain embodiments such methods comprise providing a composition comprising a plurality of supramolecular assemblies loaded with cargo, each assembly comprising any of the supramolecular assemblies and his-tagged cargo described herein and administering the cargo of the supramolecular assemblies to a subject. Administering can comprise intravenous or subcutaneous injection of the cargo into the subject in certain embodiments.

Optionally, such methods can comprise releasing the cargo from the supramolecular assemblies through facile dissolution prior to administering, wherein the released cargo substantially retains its functional 3D native structure. The releasing step can be performed by, for example, applying a chelator to the composition of supramolecular assemblies.

In certain embodiments, the supramolecular assemblies incorporating the cargo are administered to the subject to affect a prolonged release of the therapeutic agent.

Still further, the method can further comprise storing the composition of supramolecular assemblies for a prolonged time at room temperature, wherein the cargo substantially retains its functional 3D native structure.

Methods for determining tertiary structure of a protein are also provided, such methods comprising exposing a histidine-tagged (His-tagged) protein to a set of peptide units, each peptide unit of the set comprising a trimeric coiled-coil peptides each comprising a first metal-binding ligand fused to an N-terminus of a trimeric variant of a GCN4 peptide and a second metal-binding ligand fused to a C-terminus the trimeric variant of a GCN4 peptide, and one or more metal ions; wherein the one or more metal ions link with the NTS ligand and/or His₂ ligand of the peptide units to form a 3D crystal, and the his-tagged protein is reversibly incorporated into and independently organized within or on the 3D crystal, with the incorporated protein retaining a functional 3D native structure; and determining the tertiary structure of the incorporated protein. The determining step can, in some embodiments, be performed using x-ray diffraction or x-ray crystallography.

### DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings, wherein:

FIG. 1A shows a trimeric variant GCN4-p2L peptide (comprising SEQ ID NO: 1) of the present disclosure with metal-binding ligands.

FIG. 1B shows a helical wheel representation of the trimeric GCN4 peptide (SEQ ID NO: 1) coiled coil.

FIG. 1C shows a representation of assembly of the variant GCN4-p2L upon the addition of Zn²⁺ into the hexagonal crystals visualized with scanning electron microscopy (SEM).

FIG. 1D shows an image of the resulting hexagonal crystals visualized with SEM.

FIG. 1E shows a schematic representation of a metal-triggered head-to-tail assembly of variant GCN4-p2L trimeric units through metal-mediated interactions between adjacent coiled coil peptides with N-terminal nitroilotriacetic acid (NTA) moieties and C-terminal histidine (His) residues with additional interstrand interactions.

FIG. 2 shows a sequence alignment of variant peptide portions GCN4-p2L (SEQ ID NO: 1) (encoding variant GCN4 peptide portion 102) and SEQ ID NO: 2 encoding an additional embodiment of a variant peptide portion of a peptide unit 100. FIG. 2 further shows SEQ ID NOS: 3-6, which are each variants of SEQ ID NOS: 1 or 2 as described herein.

FIGS. 3A-3C show SEM of assemblies formed from the GCN4-p2L variant of FIG. 1A under various conditions, with FIG. 3A showing hexagonal 3D peptide crystals with lengths of about 5 μm that formed from combining Zn²⁺, Co²⁺ and Cu²⁺ (0.4 eq), each independently, with the GCN4-p2L variant (1 mM) for 30 minutes, FIG. 3B showing nanospheres formed from combining NiCl₂ (0.4 mM) with GCN4-p2L variant (1 mM) for 30 minutes, and FIG. 3C shows hexagonal discs formed from the combination of 0.1 mM ZnCl₂ to 1 mM of the GCN4-p2L variant for 30 minutes, noting that crystal morphology in FIG. 3C was controlled by varying the peptide to metal ratio, wherein hexagonal discs formed at a 1:10 ratio and hexagonal rods formed a 1:1 ratio.

FIG. 4 shows a representation of the X-ray structure of the head-to-tail arrangement of the GCN4-p2L variant.

FIG. 5 shows the GCN4-p2L variant trimers in a hexagonal honeycomb lattice.

FIGS. 6A-6C show images of dual strategies for incorporation His-tagged fluorophores into the GCN4-p2L peptide, with FIG. 6A showing the end of the crystals lit up, FIG. 6B showing within the growing crystals lit up, and FIG. 6C showing a combination of the approaches.

FIGS. 7A-7D show images of the incorporation of His-tagged proteins into hexagonal cyrstams, with FIG. 7A showing a schematic representation of the incorporation of an enhanced GFP with an N-terminal His₆-tag (EGFP) and FIG. 7C showing a schematic representation of the incorporation of mCherry. FIG. 7B shows Brightfield (left) and fluorescence (right) confocal images of crystals formed from the variant GCN4-2pL peptide (1 mM) incubated with ZnCl₂ (1 mM) and EGFP (7.0 μM), and FIG. 7D shows Brightfield (left) and fluorescence (right) confocal images of crystals formed from the variant GCN4-2pL peptide (1 mM) incubated with ZnCl₂ (1 mM) and mCherry (7.0 μM).

FIG. 8 is a SAX/WAXS profile of variant GCN4-2pL peptide crystals with (B) and without (A) EGFP (0.007 mM) included. The table shows q values of peaks in each spectrum.

FIG. 9A is a schematic of His-tag GFP binding to exposed NTA/M²⁺ on the P3 surface of the growing GCN4-2pL crystal.

FIG. 9B is a depiction packing of GCN4-2pL (cyan/lighter) from the X-ray structure of the crystals (cyan/lighter) demonstrating overgrowth of the coiled coils upon GFP (purple/darker) inclusion.

FIG. 9C is a graph of the intensity data of two photon excitation (800 nm) fluorescence signal of various GCN4-p2L/EGFP crystals as a function of the angle of polarization of fluorescence emission. An angle of zero corresponds to the same angle as the incident laser polarization. Each set of values corresponds to a single crystal.

FIGS. 10A-10E relate to data regarding the simultaneous incorporation of two fluorescent proteins within a GCN4-2pL crystal, with FIG. 10A showing green fluorescence, FIG. 10B showing red fluorescence, and FIG. 10C showing green and red overlay confocal images of crystals formed by GCN4-2pL (1 mM) with EGFP (1.4 μM) and mCherry (5.6 μM) and ZnCl₂ (1 mM) in MOPS buffer (20 mM, pH 7.1). FIG. 10D shows reflection interference contrast microscopy (RICM), FRET channel, and calculated FRET channel images of crystals formed by GCN4-p2L (1 mM) with given concentrations of mClover3 and mRuby3 in ZnCl₂ (1 mM) in MOPS buffer (20 mM, pH 7.1). FIG. 10E is a graph of calculated FRET sensitized emission of populations of crystals with the designated ratio of mRuby3:mClover3, with error bars from standard error of the mean.

FIGS. 11A-11C are fluorescent confocal images of GCN4-2pL crystals treated with NiCl₂ for 1 hour followed by treatment with EGFP (7.0 μM) (FIG. 11A) or mCherry (7.0 μM) (FIG. 11B) for 12 hours. FIG. 11C is a fluorescence overlay confocal image of GCN4-p2L/EGFP crystals treated with NiCl₂ for 1 hour followed by mCherry for 12 hours. Green fluorescence is labeled as G and red fluorescence is labeled as R.

FIGS. 12A-12C show stability data of EGFP within the crystals. FIG. 12A is a confocal image of crystals before and after incubation at 100° C. for 1 hour using the same laser intensity. FIG. 12B is a graph of the percentage of fluorescence retained by EGFP-retained in crystals or in solution upon incubation at 70° C. over time. FIG. 12C is a picture of EGFP in 1×PBS (0.007 mM) illuminated by 365 nm light before and after being incubated at 100° C. for 1 minute.

FIGS. 13A-13B are Brightfield (left) and fluorescence (right) confocal microscopy images of crystals formed in the presence of or treated with Pos9GFP (0.007 mM).

FIG. 14 are Brightfield (left) and fluorescence (right) confocal microscopy images of crystals treated with Pos9GFP (0.014 mM).

FIG. 15 are SEM (left) and confocal images (Brightfield and fluorescence overlay, right) of crystals formed in the presence of Pos9GTP (0.028 mM).

FIGS. 16A-16B are Brightfield (left) and fluorescence (right) confocal microscopy images of crystals formed in the presence of, or treated with, Neg30GFP (0.007 mM).

FIGS. 17-19 are SEM and confocal images of crystals formed using the metal-free methods hereof.

While the present disclosure is susceptible to various modifications and alternative forms, exemplary embodiments thereof are shown by way of example in the drawings and are herein described in detail.

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims. As previously noted, while this technology may be illustrated and described in one or more preferred embodiments, the assemblies, compositions, and methods hereof can comprise many different configurations, forms, materials, and accessories.

Generally, the supramolecular assemblies, compositions and methods hereof relate to three-dimensional (3D) crystals that self-assemble from a set of peptide units and reversibly incorporate at least one histidine-tagged (His-tagged) cargo (for example and without limitation, a protein, fluorescent molecule, and/or an oligonucleotide) within or on the crystal. As used herein, the terms “guest,” “cargo” and “guest cargo” are used interchangeably to mean and include the His-tagged proteins, compounds, and other units reversibly incorporated into the 3D crystal as a guest. In certain embodiments, the formed supramolecular assemblies comprise at least a first set of peptide units, each of which comprises a trimeric coiled-coil peptide comprising at least a variant of a GCN4 leucine zipper sequence (comprising, for example, at least 60% sequence identity thereto). In certain embodiments, the supramolecular assemblies, compositions and methods further comprise one or more metal ions to promote self-assembly of the 3D crystal, as well as one or more metal-binding ligands fused to the peptide portion of the GCN4 leucine zipper sequence variant. The morphology of the resulting crystals can vary based on a variety of factors such as, for example, the peptide to metal ratios used. In certain embodiments, the resulting crystals are hexagonal (e.g., have an open packed hexagonal arrangement). In certain embodiments, the crystals are hexagonal discs, nanospheres, or hexagonal rods.

The assemblies described herein can incorporate and overgrow the His-tagged cargo/guests within a 3D matrix of growing coiled-coil peptide crystals with high levels of efficiency. Additionally, the assemblies allow for the reversible incorporation of a variety of guest cargo that retains its functional 3D native structure (e.g., tertiary structure) and associated functional activity while incorporated within the 3D crystalline matrix. Such cargo is independently organized within or on the 3D crystal (i.e. display ordering within the crystal hosts) and, indeed, can be packed in very close proximities (e.g., as close as 6 nm) without significant disruption to the overall packing of the peptide host.

Furthermore, the cargo can display remarkable thermal stability to denaturation over extended periods of time (e.g., days) at both room temperature (RT) and extreme temperatures when within the supramolecular assemblies, which can ultimately provide prolonged storage and/or transport solutions for thermally sensitive biopolymers and other guests within a 3D crystalline matrix. The cargo can also be quickly and easily extracted from the 3D crystals while retaining its functional 3D native structure. The characteristics of the supramolecular assemblies could have far-ranging applications, from RT transport and storage of biopharmaceuticals to protein arrays for structural elucidation.

Certain conventional approaches to ordering proteins, in particular, have focused on incorporating His-tagged proteins into two dimensional (2D) arrays on nickel-nitrilotriacetic acid (Ni-NTA) surfaces for applications in high-throughput screening and cell culture. 3D crystals have also been explored for encapsulating proteins; however, it has proven difficult to achieve a high degree of ordered crystal growth, high loading levels of the cargo, and/or to incorporate large proteins as cargo using conventional techniques. For example, α-lactose crystals have been investigated as a potential host for green fluorescent protein (GFP). See, e.g., Kurimoto et al., Kinetic Stabilization of Biopolymers in Single-Crystal Hosts: Green Fluorescent Protein in α-Lactose Monohydrate, J. Am. Chem. Soc. 1999, 121(29): 6952-6953 and Shtukenberg et al., Incorporation of Macromolecules into α-Lactose Monohydrate Crystals, Crystal Growth Des 2016, 16(8): 4589-4598. GFP was found localized in a specific growing face of the lactose crystals at a modest encapsulation level (1:10⁶, GFP:lactose), with somewhat enhanced protein stability at elevated temperatures (60° C. for 1 hour). See Kurimoto supra page 3.

Protein crystals have also been used as hosts for other proteins. For instance, crosslinked 3D crystals of the Campylobacter jejuni protein (CJ) contain large pores that have been used to encapsulate horse radish peroxidase (HRP). Hamley, Protein Assemblies: Nature—Inspired and Designed Nanostructures, Biomacromolecules 2019, 20(5): 1829-1848. There, HRP displayed higher activity within the crystals at an elevated temperature (45° C.) than at room temperature, presumably due to increased substrate penetration into the crystals. Kowalski et al., Porous Protein Crystals as Scaffolds for Enzyme Immobilization, Biomater Sci. 2019, 7(5): 1898-1904. The supramolecular assemblies, compositions, and methods provided herein bridge conventional 2D and 3D approaches and provide novel and unexpected benefits and advantages when taken alone. The various aspects of the assemblies, compositions, and methods hereof are described in detail below.

Assemblies

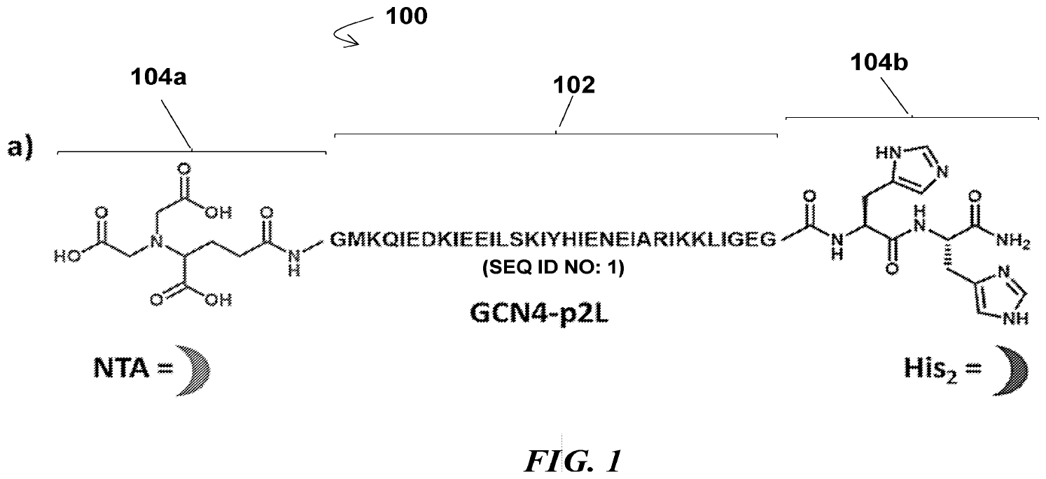

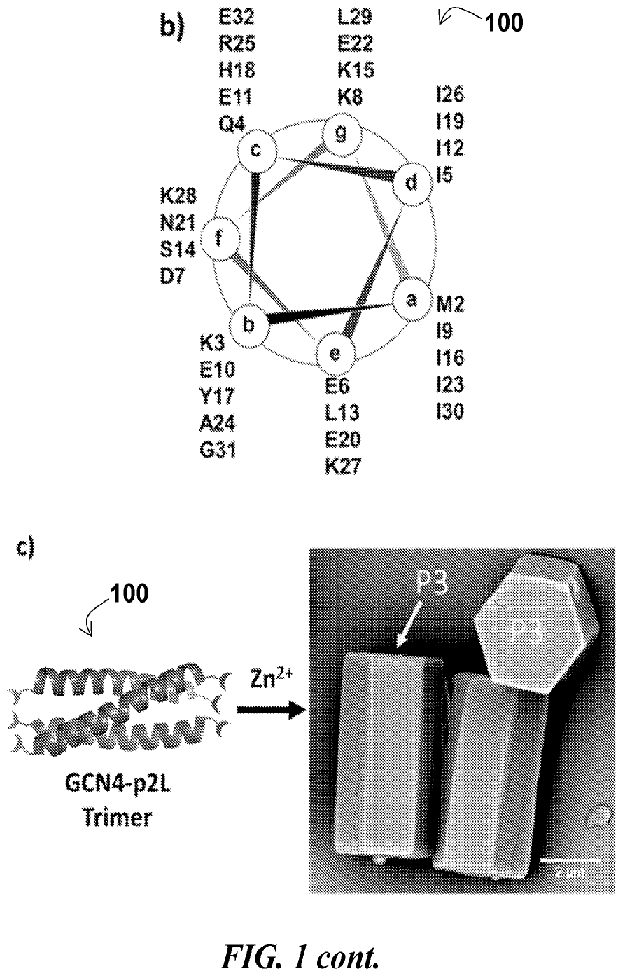

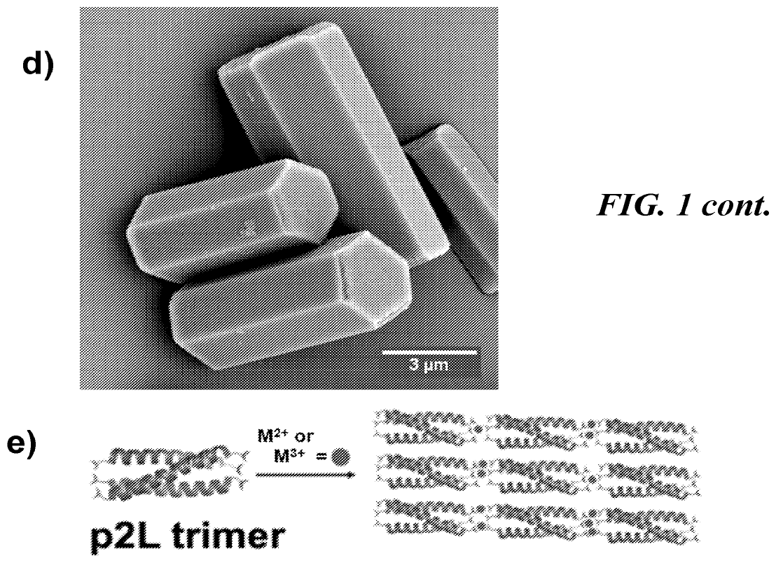

The supramolecular assemblies (e.g., 3D crystals) of the present disclosure are formed (e.g., self-assembled) of one or more building blocks, that is, in certain embodiments, at least a first set of trimeric coiled-coil peptide units. The points of extension of each building block define a geometric building unit that is equivalent to augmenting anode in an infinite 3D network and thereby becomes a means of designing and generating an inorganic, organic, or metal-organic material. As described in additional detail below, in certain embodiments, the assembly comprises a plurality of peptide units each comprising a ligand-modified trimeric coiled coil peptide 100 (see, e.g., FIGS. 1A and 1C). Additionally, in certain embodiments, each building block can further comprise one or more metal ions (see, e.g., FIG. 1E, with the metal ions are identified as M²⁺ or M³⁺).

The peptide units 100 of the supramolecular assembly comprise a trimeric coiled-coil peptide variant based on a GCN4 leucine zipper sequence (a trimeric variant of GCN4 102). For reference, a GCN4 leucine zipper is a peptide corresponding to the leucine zipper of transcription factor GCN4 (GCN4-p1) that adopts a parallel, dimeric coiled coil. The conserved leucines, like the residues of the alternate hydrophobic repeat, make side-to-side interactions (as in a handshake) in every other layer of the dimer interface. Subtle modifications to the hydrophobic residues of GCN4-p1, such as changing leucine residues to isoleucine or valine residues to isoleucine/leucine, can modulate the oligomerization of the coiled coil from dimeric to trimeric (GCN4-p2) and tetrameric (GCN4-p3) species.

A peptide unit 100 can optionally comprise one or more metal-binding ligands 104 fused with the trimeric variant of GCN4 102 to facilitate metal-promoted assembly. Non-limiting examples of such metal-binding ligands shown in FIG. TA include nitrolotriacetic acid (NTA) and/or di-histidine (His₂). These metal-binding ligands 104 can be attached at either or both ends of the GCN4 leucine zipper sequence 100 (i.e. at an N-terminus and/or a C-terminus thereof). Any metal-binding ligands now known or hereinafter developed or identified can be employed with the present supramolecular assemblies to the extent they allow for assembly of the 3D crystalline matrix and the reversible incorporation of His-tagged cargo into the 3D crystal in an ordered manner.

FIG. 1A shows at least one exemplary embodiment of a peptide unit 100 comprising a trimeric variant of a GCN4 peptide 102 with metal-binding ligands 104 attached thereto. In at least one embodiment of such peptide unit 100 (termed the “GCN4-p2L variant”, “GCN4-p2L”, or simply “p2L”), the trimeric variant of GCN4 102 is encoded by SEQ ID NO: 1, NTA (i.e. a first metal-binding ligand 104a) is fused to a first end of the trimeric variant of GCN4 102, and His₂ (i.e. a second metal-binding ligand 104b) is fused to a second end of the trimeric variant of GCN4 102. In certain embodiments, the first end of the trimeric variant of GCN4 102 is a N-terminus and the second end of the trimeric variant of GCN4 is a C-terminus. FIG. 1B shows a schematic helical wheel representation of a peptide unit 100 comprising GCN4-p2L.

The trimeric variant of the GCN4 peptide 102 portion of the GCN4-p2L variant need not be identical to SEQ ID NO: 1. Indeed, certain embodiments of the peptide units 100 can comprise other variants of the GCN4 peptide 102 or other peptides. Among other things, modifications to the starting GCN4 peptide 102 sequence portion of the peptide unit 100 can allow for and/or optimize the inclusion of differently charged cargoes (e.g., proteins and other biopolymers including, for example, supercharged proteins and the like) within the 3D crystalline matrix of the assembly. In certain embodiments, the peptide portion of the peptide unit 100 can comprise the trimeric variant of the GCN4 peptide 102 sequence having at least 60% sequence identity or more, at least 65% sequence identity or more, at least 70% sequence identity or more, at least 75% sequence identity or more, at least 85% sequence identity or more, at least 90% sequence identity or more, or at least 95% sequence identity or more to SEQ ID NO: 1. In certain embodiments, such a variant is encoded by SEQ ID NO: 2 or a sequence having at least 60% sequence identity or more, at least 65% sequence identity or more, at least 70% sequence identity or more, at least 75% sequence identity or more, at least 85% sequence identity or more, at least 90% sequence identity or more, or at least 95% sequence identity or more to SEQ ID NO: 2, including without limitation SEQ ID NOS: 5 and 6 (each peptide unit 100 comprising a peptide variant encoded thereby a “TriNL variant” or “TriNL”). Notably, and as is described in further detail below, peptide units 100 comprising the TriNL variant (including, for example, those encoded by SEQ ID NO: 2 or sequences having at least 60% sequence identity or more to SEQ ID NO: 2, including SEQ ID NOS: 5 and 6) need not necessarily comprise metal-binding ligands 104.

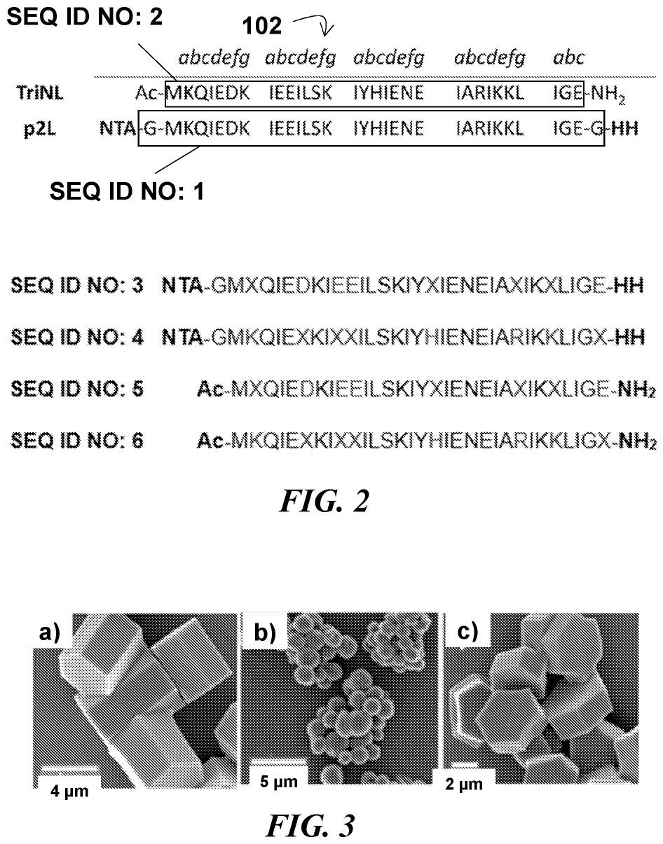

FIG. 2 illustrates the high degree of alignment between SEQ ID NOS: 1 and 2 (i.e. TriNL to p2L). As shown in FIG. 2, the TriNL variant can further comprise N-terminal acetylation and a C-terminal amino acid. In such embodiments, metal is not required to promote assembly and incorporation of cargo.

Additional variants are also contemplated. SEQ. ID NOS: 3-6 shown in FIG. 2 illustrate additional variants of the GCN4 peptide 102 sequence portion of the peptide unit 100 that may be advantageous for certain targeted cargoes.

SEQ ID NO: 3 is a variant of SEQ ID NO: 1, wherein each “X” at positions 3, 18, 25, and 28 is individually selected from the group consisting of N, Q, and E.

SEQ ID NO: 4 is also a variant of SEQ ID NO: 1, except in SEQ ID NO: 4 each “X” at positions 7, 10, 11, and 32 is individually selected from the group consisting of N, Q, R, and K.

As shown in FIG. 2, both SEQ ID NOS: 3 and 4 can be modified at an N-terminus end with NTA and at a C-terminus end with His². SEQ ID NO: 5 is a variant of SEQ ID NO: 2, wherein each “X” at positions 3, 18, 25, and 28 is individually selected from the group consisting of N, Q, and E.

SEQ ID NO: 6 is also a variant of SEQ ID NO: 2, except in SEQ ID NO: 6 each “X” at positions 7, 10, 11, and 32 is individually selected from the group consisting of N, Q, R, or K.

Both SEQ ID NOS: 5 and 6 can comprise N-terminal acetylation and a C-terminal amino acid.

The assemblies serve as hosts for encapsulated His-tagged cargo reversibly integrated and isolated within the resulting 3D crystal. Surprisingly, and as supported in the examples below, the supramolecular assemblies hereof can not only incorporate at high loading levels a variety of cargo in an ordered manner, the loading does not result in a detrimental effect on self-assembly or crystal formation even where large molecules and/or compounds are incorporated as cargo. Further, the incorporated cargo retains its functional 3D native structure and/or configuration when loaded into the crystalline matrix of the supramolecular assembly. For example, where the His-tagged guest/cargo is a protein, when incorporated into the 3D crystalline matrix, the cargo/protein retains its native tertiary fold structure and, thus, its functional activity (i.e. the loaded cargo is a His-tagged, fully folded protein).

In certain embodiments, the cargo comprises a protein, a protein complex, and/or a fusion protein. For example, and without limitation, the cargo can comprise a negatively charged protein (e.g., a slightly negatively charged protein). As used herein, a “negatively charged protein” includes naturally negatively charged proteins, to engineered supemegatively charged proteins (e.g., supemegatively charged GFP), to proteins that bind nucleic acids and form negatively charged protein:nucleic acid complexes (e.g., Cas9 proteins and variants and fusions thereof), or to protein fusions in which a protein to be delivered is associated with a negatively charged protein. Various other proteins can be used as a cargo in the present assemblies as well including, without limitation, enzymes, transcription factors, genome editing proteins, nucleases, binding proteins (e.g., ligands, antibodies, antibody fragments, nucleic acid binding proteins, etc.), structural proteins, and therapeutic proteins (e.g., tumor suppressor proteins, therapeutic enzymes, growth factors, growth factor receptors, transcription factors, proteases, etc.), as well as variants and/or fusions of such proteins.

In certain embodiments, the cargo can comprise a positively charged protein.

In certain embodiments, the cargo is a His-tagged fluorescent molecule (e.g., a fluorophore such as fluorescein) or a His-tagged oligonucleotide (e.g., a DNA molecule or an RNA molecule (including, without limitation, a small interfering RNA (siRNA) molecule)). In certain embodiments, the cargo comprises an enhanced green fluorescent protein with an N-terminal His₆-tag (EGFP) and/or a His-tagged a monomeric red fluorescent protein such as mCherry.

In certain embodiments, the cargo is a therapeutic agent, compound that comprises a drug moiety or the like, or a pharmaceutically acceptable salt of such a compound or drug moiety. The term “therapeutic agent” is intended in its broadest meaning to include a compound, chemical substance, microorganism or any agent that is capable of producing an effect in a subject or on a living tissue or cell when administered thereto. Thus, the term includes both prophylactic and therapeutic agents, as well as diagnostic agents and any other category of agent capable of having a desired effect. Therapeutic agents include, but are not limited to, pharmaceutical drugs and vaccines, nucleic acid sequences (such as supercoiled, relaxed, and linear DNA and fragments thereof, antisense constructs, artificial chromosomes, RNA and fragments thereof, and any other nucleic-acid based therapeutic), cytokines, small molecule drugs, proteins, peptides and polypeptides, oligonucleotides, oligopeptides, fluorescent molecules (e.g., fluorophores) and other imaging agents, hormones, chemotherapy, and combinations of interleukins, lectins, and other stimulating agents.

Similarly, “pharmaceutically acceptable salt” refers to those salts with counter ions which may be used in pharmaceuticals. Such salts may include, without limitation: (1) acid addition salts, which can be obtained by reaction of the free base of the parent compound with inorganic acids, such as hydrochloric acid, hydrobromic acid, nitric acid, phosphoric acid, sulfuric acid, perchloric acid, and the like, or with organic acids, such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid, malonic acid, and the like; or (2) salts formed when an acidic proton present in the parent compound either is replaced by a metal ion, e.g., an alkali metal ion, an alkaline earth ion, or an aluminum ion, or coordinates with an organic base, such as ethanolamine, diethanolamine, triethanolamine, trimethamine, N-methylglucamine, and the like. Pharmaceutically acceptable salts are well-known to those skilled in the art, and any such pharmaceutically acceptable salts are contemplated.

In certain embodiments, the cargo is a His-tagged pharmaceutical composition. The term “composition” generally refers to any therapeutic agent comprising more than one ingredient (e.g., a formulation of any of the above-listed examples of therapeutic agents). Such compositions can be prepared from isolated therapeutic agents or from salts, solutions, hydrates, solvates, and other forms of the therapeutic compounds.

The supramolecular assembly can optionally be loaded with two or more types of His-tagged cargo. For example, two or more different types of guests can be added to the supramolecular assemblies hereof in distinct regions within and on the surface of the crystal or otherwise. In certain embodiments, at least one His-tagged protein and at least one His-tagged oligonucleotide can be added to the supramolecular assembly. In other embodiments, a first cargo comprises a His-tagged fluorescein or other fluorophore (e.g., for imaging or tracing purposes) and a second cargo comprises a His-tagged therapeutic agent.

As shown in the examples below, the His-tagged cargo of the supramolecular assemblies exhibit high loading levels within the peptide crystals in an ordered manner. In certain embodiments where the cargo comprises EGFP, the EGFP is incorporated into the 3D crystal in an ordered, hourglass pattern. In certain embodiments, the supramolecular assembly comprises about a 30-35:1 ratio of peptide units 100 to loaded cargo. In certain embodiments, the supramolecular assembly comprises about a 60-70:1 ratio of peptide units 100 to loaded cargo. In certain embodiments, the supramolecular assembly comprises about a 200:1 ratio of peptide units 100 to loaded cargo. In certain embodiments, the supramolecular assembly comprises about a 30-200:1 ratio of peptide units 100 to loaded cargo.

While loaded, the cargo displays notable thermal stability (in some cases, over an extended period of time and whether at room temperature or under extreme temperature conditions). As used herein, “room temperature” means within a range of about 20-25° C. (68-77° F.). In certain embodiments, the cargo comprises a protein and, when the supramolecular assembly is stored at room temperature, the protein does not undergo substantial denaturation of its tertiary structure nor exhibit loss of functional activity that is native to the protein.

It is also noteworthy that, while stable when loaded, the cargo is reversibly incorporated within the supramolecular assembly. In other words, the loaded cargo can be easily released from the crystalline matrix of the assembly through facile dissolution with, for example, application of chelators. Other means for releasing the cargo from the matrix may become apparent to those of ordinary skill in the art in view of the disclosure and data set forth herein and such means are also encompassed within this disclosure. Additionally, and notably, when released, the cargo retains its functional 3D native structure. This can be especially advantageous where the supramolecular assembly is used to store and/or transport active compounds that may be sensitive to temperature such as, for example, pharmaceutical compounds and other therapeutic agents.

Assembly and Methods Therefore

Now referring to assembly, the supramolecular assemblies hereof are capable of self-assembly into a highly ordered crystalline matrix. The crystal morphology can vary based on various factors including, for example, the peptide to metal ratios used. In certain embodiments, the resulting crystals are hexagonal. In other embodiments, the crystals can be hexagonal discs, nanospheres, or hexagonal rods.

Crystal assembly can be metal-promoted, but depending on the composition of the peptide units, metal-binding ligands and/or metal ions are not necessarily required to load the resulting 3D crystal with cargo and achieve overgrowth.

Where a metal-mediated strategy is used to assemble the trimeric coiled coil peptide units 100, the peptide units comprise at least one metal-binding ligand (e.g., fused to a terminus of the trimer variant of a GCN4 peptide (e.g., encoded by SEQ ID NO: 1)), and one or more metal ions can be used to promote assembly into a highly ordered 3D crystalline material.

The metal ions can be divalent metal ions (M²⁺), trivalent metal ions (M³⁺), or a combination of divalent and trivalent metal ions. In certain embodiments, the one or more metal ions are selected from the group consisting of Ni²⁺, Zn²⁺, Cu²⁺, CO²⁺, Fe²⁺, Co³⁺, Fe³⁺, Rh³⁺, Ru³⁺, and Gd³⁺. A variety of metal ions useful to promote assembly may become apparent to one of ordinary skill in the art in view of the present disclosure and any such metal ions can be employed. Further, it will be understood that the metal ions can be added to initial formulations of the component parts as a salt.

An X-ray analysis of a supramolecular assembly is shown in FIG. 1C, which shows hexagonal packing of the coiled coil trimers with the ligands directed towards the growing P3 face. The ligands within and at the ends of the P3 face of each crystal can be used to bring His-tagged fluorescein (or other cargoes) within the crystal and/or to attach at the surface of the P3 face of the crystal in a metal-dependent fashion. Indeed, an appealing aspect of certain embodiments of the GCN4-p2L coiled coil hexagonal crystals is the presence of free metal-binding ligands both on the surface (e.g., the P3 face) and within the crystals, which allows His-tagged cargo to be incorporated within the crystal host.

Self-assembly (e.g., in situ) occurs, at least in part, by the assembly of a metal-binding ligand 104 and the metal(s) (or metal ion(s)). In certain embodiments, one or more metal ions are linked to the metal-binding ligands of each peptide unit, with the linkages comprising coordinate covalent bonds, noncovalent bonds, or a combination thereof (depending, for example, on the characteristics of the ligands and metals employed). FIG. 1E shows a schematic representation of this metal-triggered head-to-tail assembly (showing p2L as a representative example) for incorporating His-tagged cargo on or in 3D crystals using a metal-promoted assembly method.

Additional embodiments provide supramolecular assemblies and methods of forming the same in the absence of metal or metal ions. In such embodiments, the supramolecular assembly comprises a first set of peptide units and a second set of peptide units. The first set of peptide units can comprise any of the peptide units described herein that include one or more metal-binding ligands. For example, the first set of peptide units can comprise a trimeric variant of GCN4 such as p2L or a functional equivalent thereof.

The second set of peptide units also comprise trimeric coiled-coil peptides, except that the peptides of the second set do not have metal-binding ligands fused thereto. For example, the peptide units of the second set can comprise TriNL or a functional equivalent thereof.

Doping the first set of peptide units (e.g., p2L) with the second set of peptide units (e.g., TriNL) not only results in assembly of crystals loaded with ordered and functional cargo in the absence of metal, but such crystals exhibit beneficial properties equivalent to those described in connection with other embodiments of the supramolecular assemblies hereof. In addition, such metal-free assemblies also allow for the incorporation of a wider range of proteins (as compared to the metal-mediated assembly methods).

Methods for the preparation of the supramolecular assemblies described herein are also provided. In certain embodiments, such a method comprises combining a metal source (e.g., one or more metals, metal-based salts, and/or metal ions), a plurality of peptide units, and a plurality of His-tagged cargo units to generate a composition, wherein the composition is a 3D crystal, and the plurality of His-tagged cargo units are reversibly incorporated and independently organized into the 3D crystal with the incorporated cargo units retaining a tertiary structure and functional activity native to each such cargo unit.

The plurality of peptide units can be any of the peptide units described herein that comprise one or more metal-binding ligands including, for example, a peptide unit comprising a trimeric variant of a GCN4 peptide (e.g., GCN4-p2L) and a peptide unit comprising a peptide encoded by encoded by SEQ ID NO: 1 or a sequence having at least 60% sequence identity or more, 65% sequence identity or more, 70% sequence identity or more, 75% sequence identity or more, at least 85% sequence identity or more, at least 90% sequence identity or more, or at least 95% sequence identity or more to SEQ ID NO: 1.

Similarly, the metal source can comprise one or more metals, one or more metal salts, and/or one or more metal ions, and can of the metal or metal ion embodiments provided herein (e.g., divalent ions, trivalent ions, or a combination of divalent and trivalent ions).

The His-tagged cargo units likewise can comprise any of the cargo or guests described herein. In certain embodiments, the His-tagged cargo comprises a therapeutic agent. In certain embodiments, the His-tagged cargo comprises a pharmaceutical composition. In certain embodiments, the His-tagged cargo comprises a protein. In certain embodiments, the His-tagged cargo comprises a small molecule drug or drug conjugate. In certain embodiments, the cargo comprises a protein, a fluorescent molecule, an oligonucleotide, or a combination of two or more of the foregoing.

In certain embodiments of a method for the preparation of the supramolecular assembly, the metal source is omitted from the combining step and, thus, the composition. In such embodiments, the step of combining comprises combining a plurality of peptide units and a plurality of His-tagged cargo units to generate a composition. Each of the plurality of peptide units and the plurality of His-tagged cargo units can be any of the embodiments described herein, respectively; notwithstanding that the plurality of peptide units comprises a first set of peptide units and a second set of peptide units. Consistent with the metal-free supramolecular assembly embodiments described herein, each peptide unit of the first set comprises a trimeric coiled coil comprising a first metal-binding ligand fused to a first end of a trimeric variant of a GCN4 peptide and a second metal-binding ligand fused to a second end of the trimeric variant of a GCN4 peptide; for example, a peptide unit comprising a trimeric variant of a GCN4 peptide (e.g., GCN4-p2L) and a peptide unit comprising a peptide encoded by SEQ ID NO: 1, SEQ ID NO: 3, SEQ ID NO: 4, or a sequence having at least 60% sequence identity or more, 65% sequence identity or more, 70% sequence identity or more, 75% sequence identity or more, at least 85% sequence identity or more, at least 90% sequence identity or more, or at least 95% sequence identity or more to SEQ ID NO: 1, SEQ ID NO: 3, or SEQ ID NO: 4. Additionally, each peptide unit of the second set comprises a trimeric coiled-coil peptide without metal-binding ligands fused thereto; for example, a peptide unit comprising TriNL or a functional equivalent thereof and a peptide unit comprising a peptide encoded by SEQ ID NO: 2, SEQ ID NO: 5, SEQ ID NO: 6, or a sequence having at least 60% sequence identity or more, 65% sequence identity or more, 70% sequence identity or more, 75% sequence identity or more, at least 85% sequence identity or more, at least 90% sequence identity or more, or at least 95% sequence identity or more to SEQ ID NO: 2, SEQ ID NO: 5, or SEQ ID NO: 6 and comprising N-terminus acetylation and a C-terminus amino group.

In yet additional aspects, methods of using the supramolecular assemblies described herein are provided. The novel supramolecular assemblies hereof provide the ability to store and transport thermally stable cargo that retains its tertiary structure and function which could be particularly advantageous in various fields.

While specific methods of use are provided herein, other uses will be clear to those of skill in the art based on the disclosure relating to supramolecular assemblies and design, the crystallization of cargo, and cargo incorporation.

Pharmaceutical and Medical Applications

The supramolecular assemblies hereof can be particularly useful in the fields of medicine, pharmaceuticals, and biopharmaceuticals. In cases where a supramolecular assembly or compositions thereof are used in connection a therapeutic agent or pharmaceutical composition, purification and/or sterilization may be advantageous. The supramolecular assemblies can enable control over assembly through mixing of purified components in vitro. This feature, combined with the supramolecular assemblies' large capacity for incorporating cargo that retains its tertiary structure and functional activity and the thermal stability imparted to the loaded cargo, makes them well-suited for the encapsulation of a broad range of materials including small molecules, nucleic acids, and proteins, as discussed above. In turn, the supramolecular assemblies hereof could be used for many applications in medicine and biotechnology, including therapeutic agent and pharmaceutical manufacturing, the transport, storage and stabilization of biopharmaceuticals, drug delivery, and vaccine design.

The methods for preparation described herein can be applied in the context of pharmaceutical manufacturing. The supramolecular assemblies hereof can be manufactured as part of or in conjunction with a process for preparing a therapeutic agent or pharmaceutical composition. For example, compounds to be used for therapeutic administration are often stored in unit or multi-dose containers, for example, sealed glass ampules or vials. As the amount of therapeutic agent loaded into each supramolecular assembly hereof can be readily determined, the methods of the present disclosure can further comprise the step of allocating a desired dosage of therapeutic agent loaded in the supramolecular assembly composition into the appropriate storage containers to further facilitate distribution.

Accordingly, methods for treating a subject experiencing or at risk for experiencing a disease state are provided. As used herein, the terms “patient,” “subject,” and “individual” are used interchangeably. None of the terms require the supervision of medical personnel. For example, administering to an individual includes the individual administering the therapeutic agent to themselves, as well as a medical professional administering the therapeutic agent to the individual. The terms “treat,” “treating,” or “treatment” include reducing, alleviating, abating, ameliorating, relieving, or lessening the symptoms associated with a disease state in either a chronic or acute therapeutic scenario.

In certain embodiments, a method for treating a subject experiencing or at risk for experiencing a disease state comprises providing a composition comprising a plurality of supramolecular assemblies loaded with cargo and administering the cargo of the supramolecular assemblies to a subject (e.g., subcutaneously, intravenously, intramuscularly, etc.). The term “administering” generally refers to any and all means of introducing the therapeutic agent of the supramolecular assembly to the subject including, but not limited to, by oral, intravenous, intramuscular, subcutaneous, transdermal, inhalation, buccal, ocular, sublingual, vaginal, rectal, and like routes of administration.

Any of the supramolecular assemblies described herein can be employed with this method, including those that incorporate metal or metal ion(s) or those that are metal-free. Additionally, the cargo can be any of the cargo described herein or other compounds or molecules that may be subject to storage or transport prior to administration to a patient.

In certain embodiments, the method for treating can further comprise releasing the cargo from the supramolecular assemblies through facile dissolution prior to administering, wherein the released cargo substantially retains the tertiary structure and functional activity native to the cargo. For example, releasing the cargo can comprise applying a chelator to the composition of supramolecular assemblies.

In certain embodiments (e.g., for sustained release drug delivery), it may not be desirable to first release the therapeutic agent from the crystalline matrix of the assembly. Indeed, where the prolonged, slow, sustained-release of a therapeutic agent is desired, the supramolecular assemblies incorporating and/or loaded with the therapeutic cargo can be administered to the subject (e.g., directly). As the crystalline matrix will take some period of time to dissolve in vivo, it effectively functions as a sustained-release matrix thereby releasing the incorporated therapeutic agent into the subject's body over a period of time as the 3D crystal dissolves.

The method can further comprise storing the composition of supramolecular assemblies for a prolonged time at room temperature (or otherwise). Due to the novel characteristics of the supramolecular assemblies described herein, even over an extended period of time, the loaded cargo can substantially retain its tertiary structure and any functional activity native thereto.

Structural Elucidation Applications

Macromolecular X-ray crystallography applied to crystals of biological molecules enables the visualization of structures of proteins, DNA, RNA, and their complexes with near to full atomic resolution. The specific shapes and 3D structure of these molecules is often tightly related to their function and physicochemical properties. Protein modeling in particular is integral to drug development and design. However, obtaining diffraction-quality crystals can be a rate-limiting step in macromolecular X-ray crystallography. Since each sample has different and specific characteristics, crystallization conditions cannot always be predicted with certainty. As such, crystal nucleation and growth are often only enabled through tedious experimentation and screening, if at all. Often, the size, shape and surface of the sample or complexes of interest are altered through genetic and biochemical manipulation to facilitate crystallization.

The supramolecular assemblies hereof can facilitate the crystallization of biological molecules that heretofore have been difficult to crystallize. As the loaded cargo retains its functional 3D native structure when incorporated into the assemblies hereof, use of the supramolecular assemblies in solving for the 3D structure of cargo would be advantageous.

Such biological molecules (e.g., a protein) could be loaded into the supramolecular assembles hereof as a cargo unit as previously described (e.g., his-tagging the molecule and combining the His-tagged molecule with the peptide units (and, in certain embodiments, a metal source). When incorporated, the tertiary structure and other characteristics of the loaded cargo could then be easily visualized/determined (e.g., via x-ray diffraction or x-ray crystallography).

### Certain Definitions

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

As used herein and in the appended claims, the singular forms “a,” “and,” and “the” include plural referents unless the context clearly dictates otherwise. Thus, for example, reference to “a compound” includes a plurality of such compounds. When ranges are used herein for physical properties, such as molecular weight, or chemical properties, such as chemical formulae, all combinations and sub-combinations of ranges and specific embodiments therein are intended to be included.

The term “about,” when referring to a number or a numerical range, means that the number or numerical range referred to is an approximation within experimental variability (or within statistical experimental error), and thus the number or numerical range may vary between 1% and 15% of the stated number or numerical range.

The term “comprising” (and related terms such as “comprise” or “comprises” or “having” or “including”) is not intended to exclude an embodiment of any compound, composition, method, process, or the like that may “consist of” or “consist essentially of” the described features. The invention illustratively described herein may be suitably practiced in the absence of any element(s) or limitation(s), which is/are not specifically disclosed herein.

“Percent (%) sequence identity” with respect to a reference to a sequence is defined as the percentage of amino acid or nucleic acid residues, respectively, in a candidate sequence that are identical with the residues in the reference sequence, after aligning the sequences and introducing gaps, if necessary, to achieve the maximum percent sequence identity and not considering any conservative substitutions as part of the sequence identity. Alignment for purposes of determining percent sequence identity can be achieved in various ways that are within the skill of the art, for instance, using publicly available computer software. For example, determination of percent identity or similarity between sequences can be done, for example, by using the GAP program (Genetics Computer Group, software; now available via Accelrys on http://www.accelrys.com), and alignments can be done using, for example, the ClustalW algorithm (VNTI software, InforMax Inc., Gaithersburg, MD). Further, a sequence database can be searched using the nucleic acid or amino acid sequence of interest. Algorithms for database searching are typically based on the BLAST software (Altschul et al., 1990), but those skilled in the art can determine appropriate parameters for aligning sequences, including any algorithms needed to achieve maximal alignment over the full length of the sequences being compared. In some embodiments, the percent identity can be determined along the full-length of the nucleic acid or amino acid sequence.

The terms and expressions, which have been employed, are used as terms of description and not of limitation. In this regard, where certain terms are defined under “Certain Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Certain Definitions,” are used in the “Detailed Description,” such use is solely for ease of reference and is not intended to limit any disclosure made in one section to that section only; rather, any disclosure made under one subheading is intended to constitute a disclosure under each and every other subheading.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

In the following description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary, but remain applicable in view of the data provided herein.

Various techniques and mechanisms of the present disclosure will sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, wherever feasible and convenient, like reference numerals are used in the figures and the description to refer to the same or like parts or steps. The drawings are in a simplified form and not to precise scale. It is understood that the disclosure is presented in this manner merely for explanatory purposes and the principles and embodiments described herein may be applied to assembly components that have configurations other than as specifically described herein. Indeed, it is expressly contemplated that the components of the assemblies, compositions, and methods of the present disclosure may be tailored in furtherance of the desired application thereof.

### EXAMPLES

The following examples serve to illustrate the present disclosure. The examples are not intended to limit the scope of the claimed invention in any way.

### Example 1: Crystal Formation

Metal-mediated strategies were used to assemble trimeric coiled coil peptide building blocks based on a trimeric variant of the GCN4 peptide (see FIG. 1B). Head-to-tail assembly of these trimeric units were achieved through metal-mediated interactions between adjacent coiled coil peptides with N-terminal NTA moieties and C-terminal histidine (His) residues to synthesize the trimer variant GCN4-2pL peptide (also referred to as “2pL” or the “2pL peptide”) with the additional interstrand interactions (see FIGS. TA, 1B and TE).

Various metal ions (0.4 mM) were independently added to 1 mM of the 2 μL peptide for 30 minutes to facilitate the formation of hexagonal 3D peptide crystals. The trimer variant GCN4-2pL peptide rapidly formed hexagonal 3D crystals with the addition of Zn²⁺, Co²⁺ and Cu²⁺ (0.4 eq). FIG. 3A shows hexagonal 3D peptide crystals with lengths of about 5 μm that formed from the addition of ZnCl₂ (0.4 eq) to the GCN4-p2L variant (1 mM) for 30 minutes. FIG. 3B shows nanospheres formed from combining NiCl₂ (0.4 mM) with the GCN4-p2L variant (1 mM) for 30 minutes. FIG. 3C shows hexagonal discs formed from the combination of 0.1 mM ZnCl₂ to 1 mM of the GCN4-p2L variant for 30 minutes (noting that crystal morphology was controlled by varying the peptide to metal ratio, wherein hexagonal discs formed at a 1:10 ratio and hexagonal rods formed a 1:1 ratio).

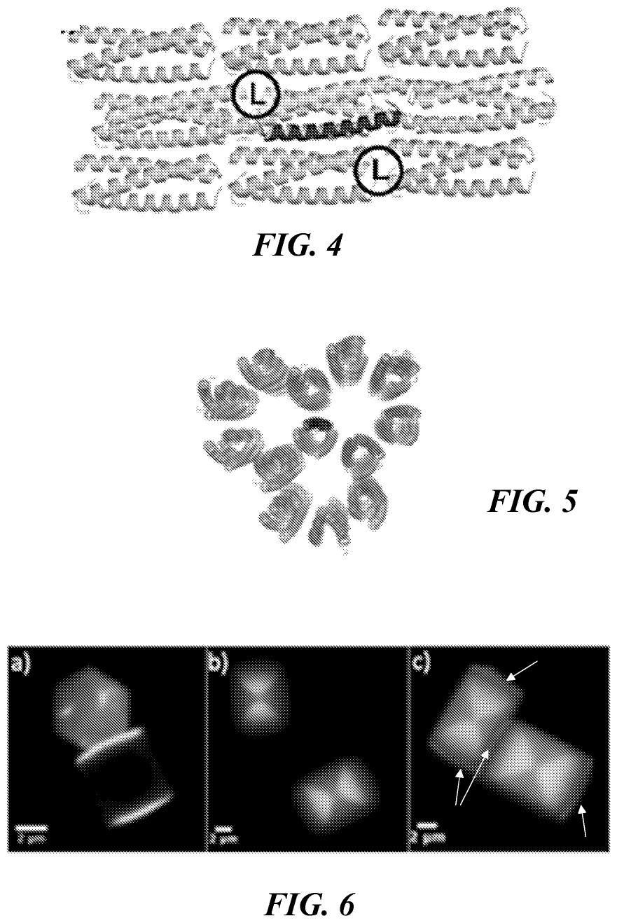

Treatment of the fully formed hexagonal crystals with ethylenediaminetetraacetic acid (EDTA) resulted in an immediate dissolution of the crystals. The crystal structure of the peptide assembly was solved to 1.9 Å resolution (PDB 5KKV), which elucidated the head-to-tail arrangement of the coiled coils (FIG. 4) and the hexagonal honeycomb, antiparallel packing in the crystal (FIG. 5).

### Example 2: Incorporation of Cargo

With the head-to-tail arrangement observed in the crystal structure of the trimeric p2L peptide, the hexagonal faces of the crystals were studied to confirm the presence of free metal-binding ligands. If present, these ligands (e.g., nitroilotriacetic acid (NTA)) could be loaded with metal ions and would be available to bind His-tagged cargoes. Accordingly, the metal-ligand interactions used to promote assembly were also harnessed to include His-tagged fluorophores (His-Fl or His-Rh) to both the interior and surface of the crystals (FIGS. 6A-6C).

The preformed hexagonal crystals were treated with NiCl₂ and fluorescein-labeled His₆ (Fl-His₆). Green fluorescence was associated with all crystals, with the fluorescence localized to the hexagonal (P3) faces of the crystal (FIG. 6A).

The inclusion of Fl-His₆ within the core of the crystals as the crystals were growing was also investigated. In this case, the fluorescence was localized to 2 sections of the crystals, forming an “hourglass” pattern that projected out towards the P3 crystalline faces (FIG. 6B).

Thereafter, the two strategies for directing guests to the coiled coil crystals were combined and green fluorescence was again observed in an hourglass pattern within the crystals, whereas red fluorescence was found at the ends of the crystals on the P3 faces (FIG. 6C; red fluorescence indicated by arrows). Overall, these His-tagged directed experiments demonstrated a facile means to direct guests to specific locations within the crystals.

### Example 3: Incorporation of Protein Cargo

As previously determined, the variant GCN4-2pL peptide hexagonal crystals have free metal-binding ligands on the surface and within the crystals, which can be used to bring His-tagged cargo within the crystals in at least a metal-dependent fashion. The size and charge of His-tagged guests that could be incorporated and overgrown within the supramolecular assemblies described herein were then explored (i.e. could larger His-tagged guests, such as proteins, be incorporated into the crystal hosts?).

Proteins with green fluorescent protein (GFP) were used as a model protein guest as the fluorescence of GFP is dependent on proper folding and its beta barrel is comparable in size to the trimeric coiled coil of GCN4-p2L which may facilitate overgrowth within the peptide crystal. Additionally, a variety of supercharged GFP mutants were developed, which allowed for analysis of how charge effects the peptide crystal host-protein interaction.

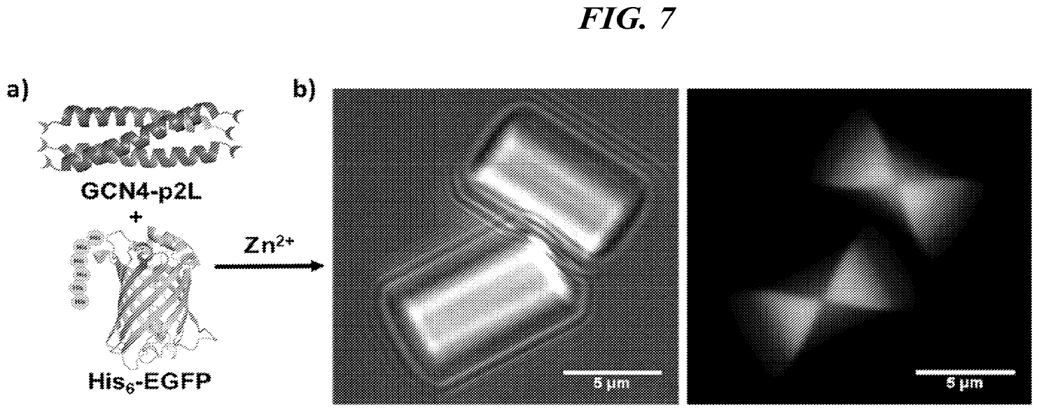

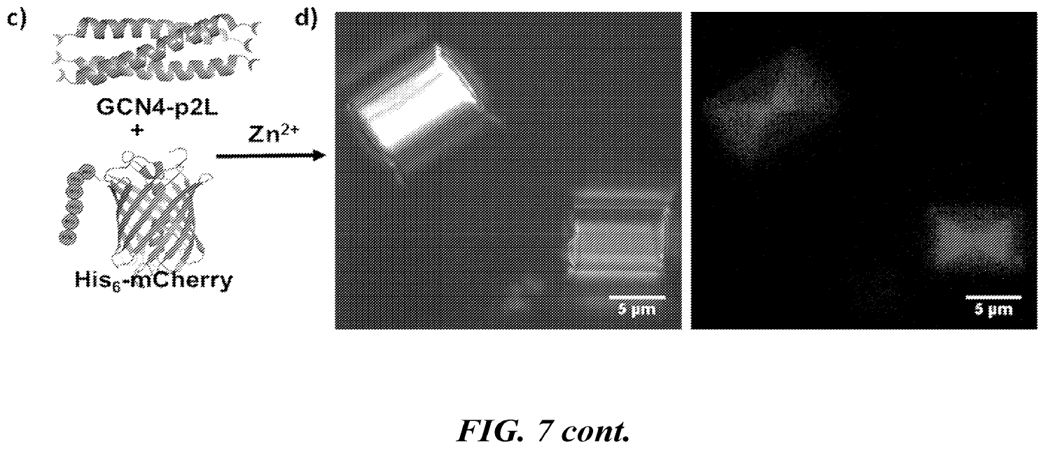

Enhanced GFP with an N-terminal His₆-tag (EGFP) was incorporated into the GCN4-p2L crystals during their growth. The variant GCN4-2pL peptide (1 mM) and ZnCl₂ (1 mM) were combined with EGFP (0.007 mM) in 3-(N-morpholino)propanesulfonic acid (MOPS) buffer (20 mM, pH 7.1) (see FIG. 7A). A precipitate immediately formed that was collected after 1 hour and washed.

Scanning electron microscopy (SEM) was used to monitor the overall morphology; crystals of about 5 μm were obtained with a hexagonal prism shape, that were similar to the crystals observed without EGFP (see FIGS. 1C and 1D).

To determine if folded EGFP was located within the coiled coil peptide crystals, fluorescent confocal microscopy was used. The resultant crystal assemblies displayed a strong green fluorescence that was mostly confined to two symmetrical sections of the crystal in an “hourglass” orientation (see FIG. 2B). This hourglass pattern would form if the His-tag of EGFP bound to available metal-charged ligands on the two symmetry-related, growing P3 faces at the ends of the crystal. As those faces grow in area, so would the subvolume associated with EGFP fluorescence.

Low levels of fluorescence were also observed outside of the hourglass regions, perhaps due to EGFP having some interactions with the coiled coil peptides themselves.

A red fluorescent protein, His-tagged mCherry, that has a similar size and charge to EGFP was also evaluated. As shown in FIGS. 7C-7D, when the crystals were formed in the presence of mCherry (0.007 mM), crystals with red fluorescence in an hourglass pattern were also obtained.

These data, in addition to the SEM, demonstrate that these protein guests do not preclude the formation of the hexagonal crystal morphology when incorporated within the crystals. Importantly, the observed fluorescence indicates that EGFP and mCherry were incorporated within the crystals in their properly folded forms, indicating that large guests, such as a protein, can bind to the P3 face and then be overgrown within the peptide crystals.

### Example 4: Measuring Levels of Protein Guest Incorporation

The levels of protein guest incorporated into the GCN4-2pL/Zn²⁺ crystals was determined to evaluate the assemblies' capabilities as vessels to hold proteins. A beneficial feature of these peptide crystals is they undergo facile dissolution under mild conditions with chelators, so as to release and quantitate the encapsulated EGFP within the crystals.

The crystals were treated with EDTA (10 mM) for 10 minutes for dissolution, and the levels of peptide and protein were quantitated by ultra performance liquid chromatography (UPLC) and fluorescence spectroscopy, respectively, using standard curves. Through this analysis, a 200:1 ratio of peptide to protein cargo, or one protein guest for every 60-70 coiled-coil units, was observed. Most of the fluorescence was localized to the hourglass region that accounted for about 50% of the total volume of the crystal. Therefore, within the hourglass section of the crystal, there was about protein guest for every 30-35 coiled-coils. This is a striking level of incorporation of the guest EGFP within the intact crystal host.

### Example 5: Internal Packing Analysis

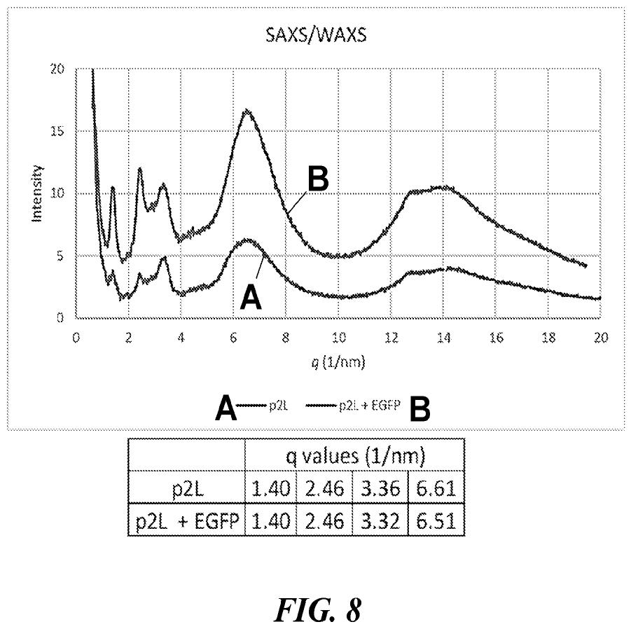

With the high level of inclusion of the guest protein that resulted, the host crystal was analyzed for signs of disruption caused by the packing of coiled coils within the crystal (as compared to like crystals without a guest). The crystals were analyzed with small- and wide-angle X-ray scattering (SAXS/WAXS) to assess their internal packing. The SAXS/WAXS profiles of the crystals with and without EGFP were very similar, with signals at the same q values (FIG. 8). These data indicate that the overall packing of the host crystals is maintained in the open packed hexagonal arrangement, and is not significantly altered by the inclusion of the EGFP protein guest.

### Example 6: Protein Organization within the Crystals

Fluorescence polarization imaging was used to study the protein organization within the crystals. If the chromophores of the fluorescent proteins aligned in mostly one orientation, as opposed to a random distribution, emission anisotropy as a function of the angle of polarization would be expected.

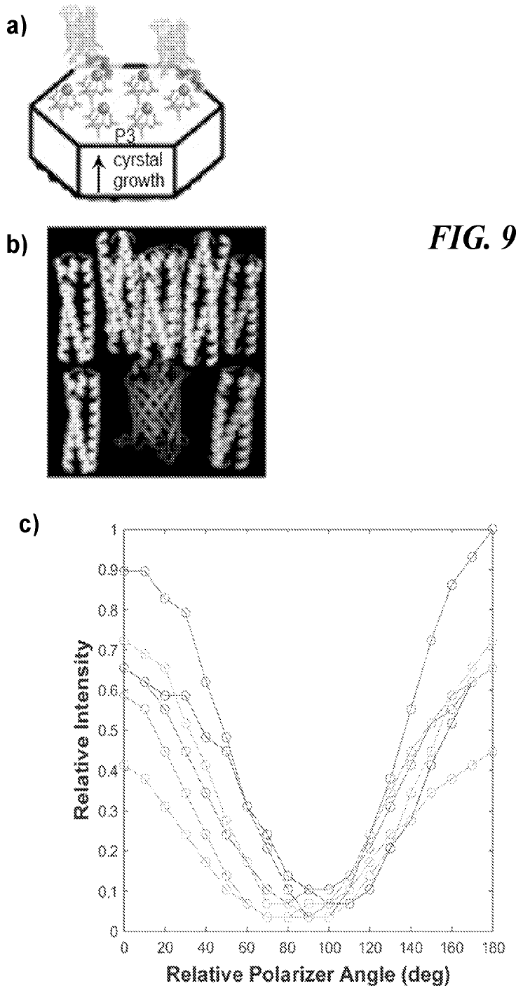

Fluorescence polarization measurements were made to determine if the GFP chromophores were oriented within the crystal using a two-photon emission fluorescence microscope with a rotating polarizer. The luminescence of the GFP-containing crystals was monitored at 510 nm with excitation light (470 nm to prevent photobleaching) that was polarized along orthogonal directions. When the GCN4-p2L crystals containing EGFP were interrogated, the intensity of the fluorescence emission was found to fluctuate as a function of the angle of polarization (FIG. 9C), indicating a similar orientation and a level of order for the EGFP guest molecules within the crystalline host. Taken together, these data support that the hexagonal arrangement of the coiled coils within the crystal was mostly maintained in the presence of protein guest, with additional order in the alignment of EGFP proteins with respect to one another.

### Example 7: Concurrent Incorporation of Guests

Simultaneous incorporation of EGFP and mCherry during GCN4-p2L crystallization provided information about the potential for concurrent protein delivery and proximities between adjacent FPs. mCherry and EGFP (7.0 μM of total protein) were initially used with a molar ratio of 5:1 of mCherry:EGFP since mCherry has a lower level of brightness.

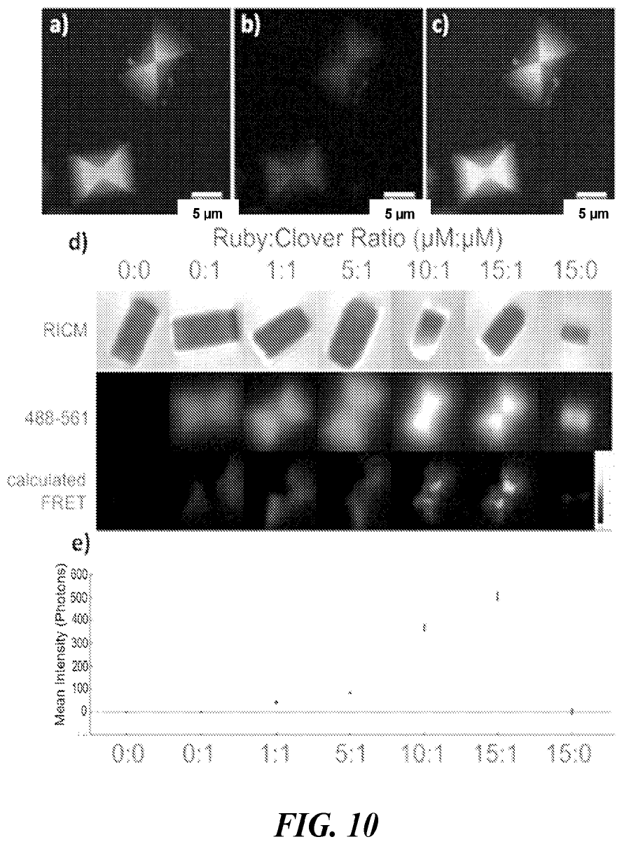

Both proteins were incorporated into single crystal lattices and imaged by confocal microscopy (FIGS. 10A-10B). Substantial overlay of green and red fluorescent signals demonstrated co-localization of the protein guests (FIG. 10C). We concluded that some of the mCherry and EGFP are within diffraction limited spacings of about 250 nm.

Based on the measured peptide to protein/guest ratio of about 35:1 within the hourglass segments of the crystals, it was investigated if the two spectrally distinct fluorescent proteins were in close enough proximity to each other to enable Forster resonance energy transfer (FRET). A FRET pair mClover3 and mRuby3 were incorporated, each with an N-terminal His-tag, into the GCN4-p2L crystals over a range of ratios, and either alone or together the same hourglass fluorescence was observed with co-localization (FIGS. 10A-10B). These fluorescent proteins exhibited enhanced brightness and have a Förster radius on the order of just over 6 nm.

To probe the inter-protein distances within the crystal by FRET, therefore, the donor, mClover3 ratio within the crystals was fixed and acceptor, mRuby3, was doped up to a 1:15 molar ratio, respectively. The resulting crystals were imaged in an epifluorescence configuration (FIG. 10D). As the incorporation of acceptor mRuby3 protein was increased, a definitive sensitized mRuby3 emission was measured. These data indicate a close packing of the guest proteins within the hexagonally packed GCN4-p2L crystalline host (FIGS. 10D-10E).

### Example 8: Harnessing Free Ligands in Fully Formed Crystals

As described above, the variant GCN4-p2L peptide crystals displayed metal-binding ligands on the P3 face. A study was performed to determine if these free ligands could be harnessed to attach His-tagged proteins to the surface of the fully formed crystals in a metal ion-dependent fashion. Thus, pre-formed crystals (1 mM GCN4-p2L: 1 mM ZnCl₂) were treated with NiCl₂ (1 mM) for 1 hour and washed. These Ni²⁺-treated crystals were then incubated with either His-tagged EGFP or mCherry (both at 7.0 μM).

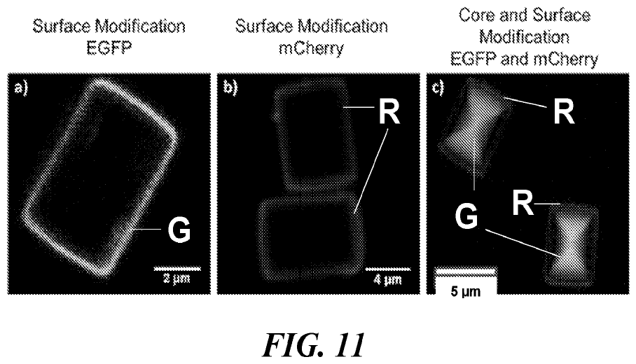

When visualized via confocal microscopy, fluorescence attributed to the proteins was observed on both of the crystal's P3 faces, as well as on the other sides of the crystals (FIGS. 11A-11B). These data suggest that there are anon-metal mediated interactions involved in protein-crystal binding. To test if metal ions are required for proteins binding to the crystal surface, we incubated pre-formed crystals with His-tagged EGFP (7.0 μM) without pre-treatment with Ni(II) ions. The resultant assemblies also fluoresced green on all sides (Figure S10) however, the level of fluorescence was noticeably lower without Ni(II) treatment. These data show that Ni²⁺ is not necessary for protein interactions with pre-formed crystals, but the addition of metal ions does maximize protein levels on the crystal surface. Similar data was observed with ZnCl₂ pre-treated as well (data not shown).

Additionally, if two different proteins could be added to the peptide crystals in two distinct regions within and on the surface of the crystal was assessed. Crystals that were formed in the presence of His-tagged EGFP were subsequently incubated with NiCl₂, followed by His-tagged mCherry. Confocal microscopy of the resulting crystals showed a green fluorescent hourglass pattern (labeled G) within the crystals as observed above with EGFP, but in this instance with noticeable red fluorescence (labeled R) on the crystal surface from mCherry (FIG. 11C).

### Example 9: Thermal Stability

Methods to enhance the room temperature (RT) storage of proteins are of interest for reasons as diverse as enzyme stability for biochemical and chemical processes to the shelf life of biopharmaceuticals. Proteins with small molecule crystals, such as phthalic acid and lactose, have demonstrated enhanced protein stability, but low loading efficiency, slow crystal growth and/or difficulties with resolubilizing these crystalline the crystal host. By contrast, the hexagonal crystals derived from the trimeric coiled coil peptide p2L can be loaded with His-tag fluorescent proteins at about 1-2% of the total crystal volume, and mild treatment with EDTA immediately dissolves the crystals and releases the protein cargo into solution. Protein aggregation can occur when a protein is partially unfolded due to thermal treatment. Therefore, isolation of His⁸-GFP within the single crystal matrix of the coiled coil peptide may constrain the mobility of the protein, thereby limiting both unfolding and aggregation.

To test the hypothesis that proteins isolated within the crystalline peptide matrix are less prone to thermally-induced unfolding and aggregation and, thus, support the crystals could serve as protein stabilization vehicles, samples of GCN4-p2L crystals with EGFP (1 mM GCN4-p2L:7.0 μM His-tagged EGFP) were heated over different time periods and at different heats (RT and elevated temperatures (37° C. and 100° C., respectively) as compared to protein solutions. GFP fluoresces only when properly folded (i.e. in its 3D functional native structure), thus providing a means to monitor protein folding both in solution and within crystals.

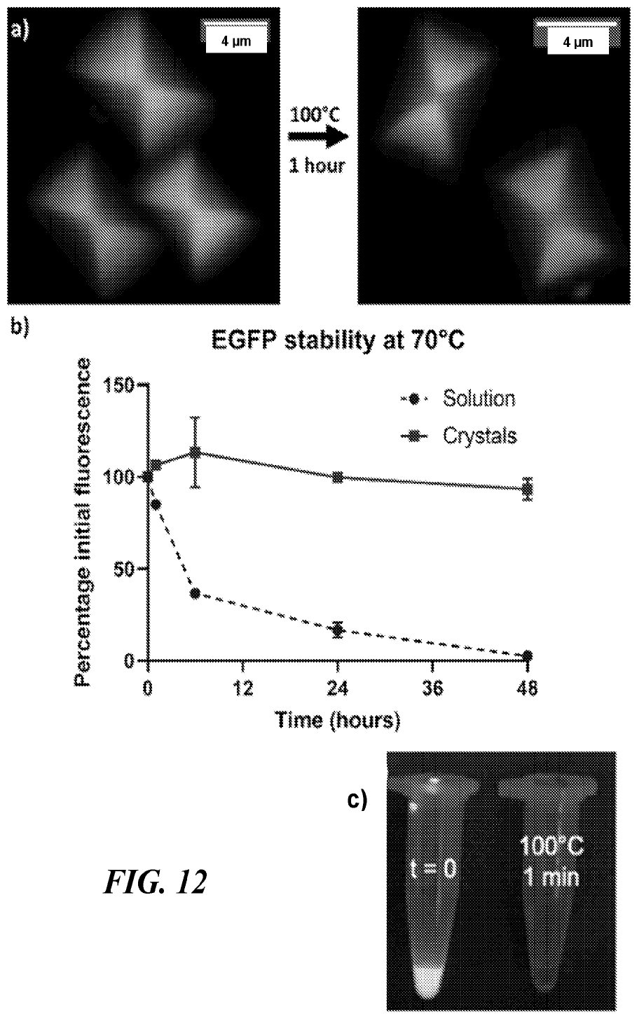

First, all GCN4-p2L crystals containing Hiss-GFP were heated to 100° C. for 1 hour. Remarkably, there was substantial fluorescence observed within the crystals (FIG. 12B), whereas solutions of Hiss-GFP when heated to 100° C. were no longer fluorescent after 1 minute (FIG. 12C). Because of their size (about 5 microns in length) and the lack of crystal aggregation, flow cytometry was used to monitor the fluorescence of populations of crystals. These data indicate the stability of the protein even under extreme temperatures while in the crystalline matrix.

Crystals containing His-tagged EGFP maintained 93% of their initial fluorescence after being incubated at 70° C. for 4 days, and after one week 70% of the fluorescence was retained. In contrast, a solution of His-tagged EGFP in PBS lost all fluorescence after incubation at 70° C. for 1 day (FIG. 12B).

### Example 10: The Role of Surface Charge of the His-Tagged Guests

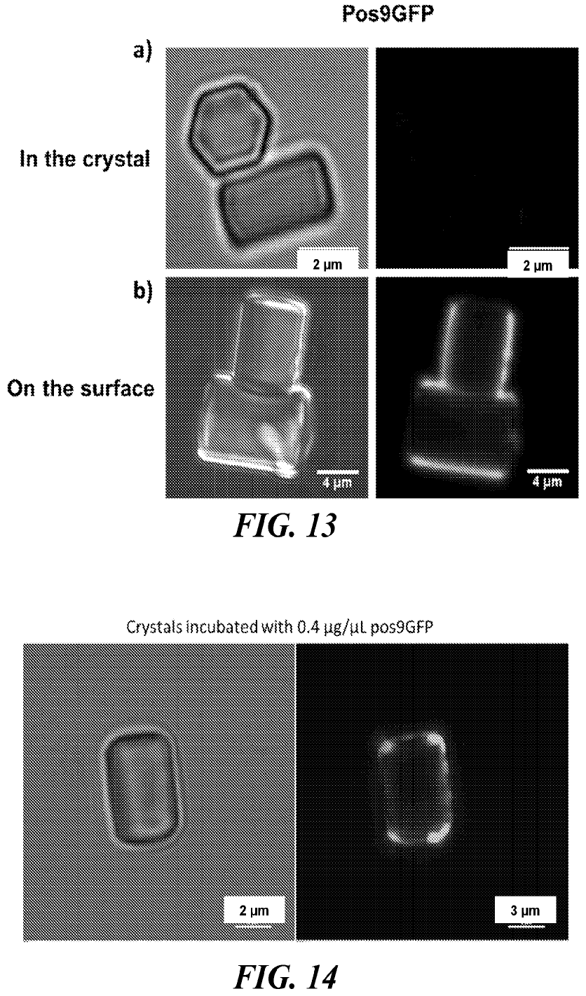

A major driving force for Hiss-GFP to occupy positions within the crystalline lattice of the coiled coil GCN4-p2L peptide is the interaction between the His-tag and exposed ligands bound to Zn²⁺ on the growing P3 face of the crystal (FIG. 13A). The His-tag is present on the N-terminus of the protein, so for the crystal to continue growing, this “defect” needs to be overgrown with additional peptide through other interactions (FIG. 13B). Mutants of Hiss-GFP with varied electrostatic surface potential were investigated, as well as “supercharged” variants of GFP wherein solvent-exposed residues are mutated to either positively or negatively charged amino acids. These supercharged GFPs can be used as building blocks for biomaterials and their charges can significantly impact the morphology and physical characteristics of the materials. As a comparison for EGFP, which has a theoretical charge of −11, whether or not two supercharged His-tagged proteins with charges of −30 (Neg30GFP) and +9 (Pos9GFP) could be incorporated into the crystals of the present disclosure was examined.

Pos9GFP was the first supercharged variant attempt at incorporating as a guest within the GCN4-p2L peptide crystals. However, attempts to incorporate Pos9GFP were unsuccessful. When ZnCl₂ (1 mM) was added to the peptide (1 mM) and Pos9GFP (0.007 mM), hexagonal peptide crystals were formed with no detectable amount of protein guest incorporated (FIG. 13A-13B). The morphology of the crystals was unchanged, but they contained no protein guest.

Additionally, no protein was incorporated when the concentration was increased to 14 or 21 μM. Once the concentration was raised to 28 μM, a very small amount of fluorescence was observed within the resultant assemblies. However, the increase in protein concentration also altered the morphology of the structures. The crystals were much smaller, and less well-defined. Additionally, there were ill-defined aggregates which contained the majority of detectable fluorescence (FIG. 15).

Mapping the charge distribution and Zeta potential measurements of pre-formed crystals showed they had an overall negative charge (−31 mV). It was then hypothesized that while Pos9GFP could not be incorporated into the crystal lattice, there may be electrostatic interactions allowing it to attach to the crystal surface. In fact, when Pos9GFP (0.007 mM) was added to pre-formed crystals treated with Ni²⁺, fluorescence was observed, but only concentrated on the sides of the crystals (FIG. 13A).

The ability to give the crystals a more positive charge would be beneficial if these materials were to be used as cell-penetrating drug delivery agents. Increasing the concentration of the protein (0.0014 mM) allowed the protein to bind to both the sides and the P3 face of the crystal, but also led to protein aggregation (FIG. 14).

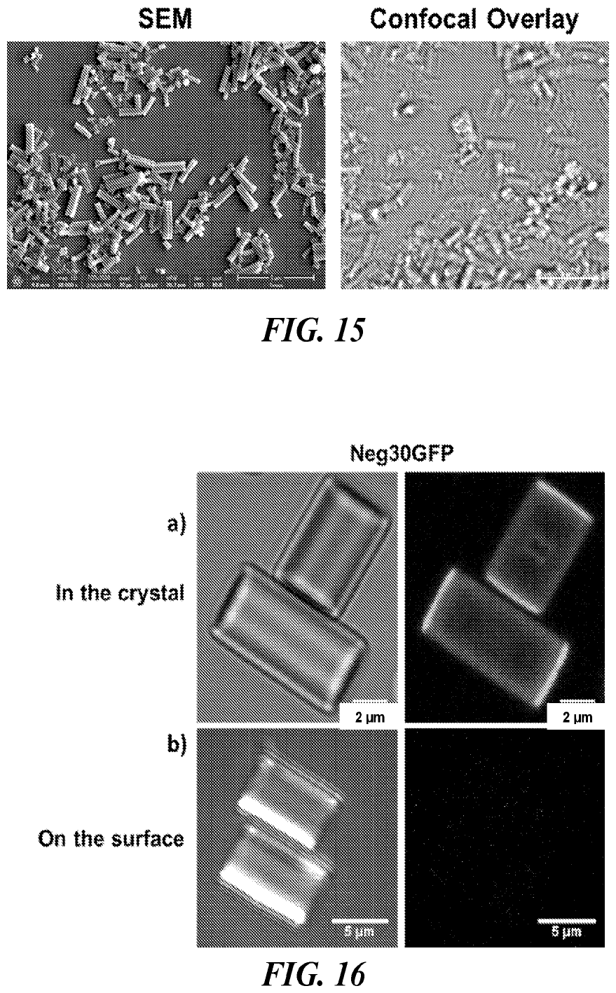

Alternatively, when Neg30GFP was added to the peptide pre-formation, it was included within the crystal matrix after assembly was initiated with ZnCl₂. However, it was localized to the outside edges of the assembly (FIG. 16B) in direct contrast to the hourglass incorporation of EGFP and mCherry. Similar to Pos9GFP, it is not included in the proposed nucleation site in the middle of the crystal, but in contrast it is included as the crystal grows longer.

Due to the negative surface charge of both unaltered crystals and Neg30GFP, it was hypothesized that they would repel each other and prevent adherence of the protein to the crystals. Indeed, when Neg30GFP is added to Ni²⁺ treated crystals, no amount of fluorescence from Neg30GFP was detected (FIG. 16A).

### Example 11: Metal Free Assembly

The methods investigated heretofore required the use of metal. Through doping the GCN4-p2L crystals with TriNL, a peptide without metal binding ligands, the crystals not only assembled in the absence of metal, but also incorporated a wider range of proteins.

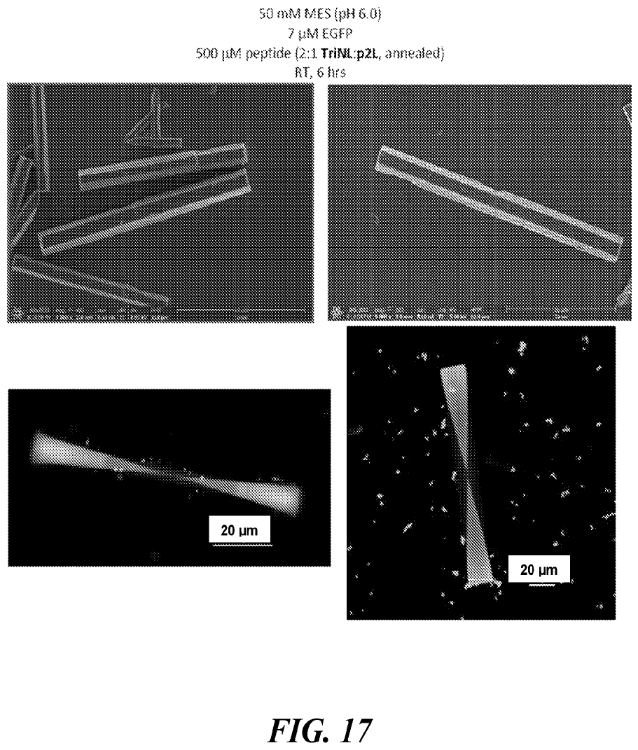

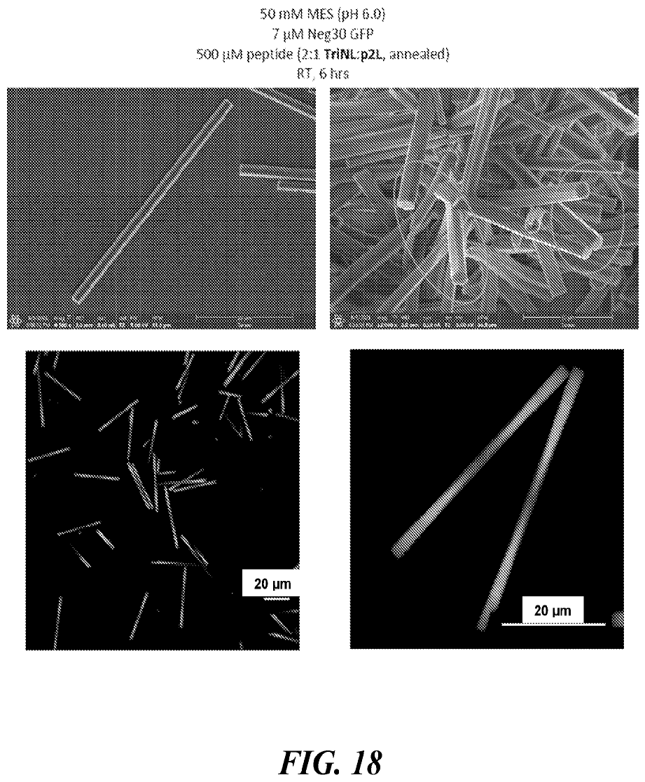

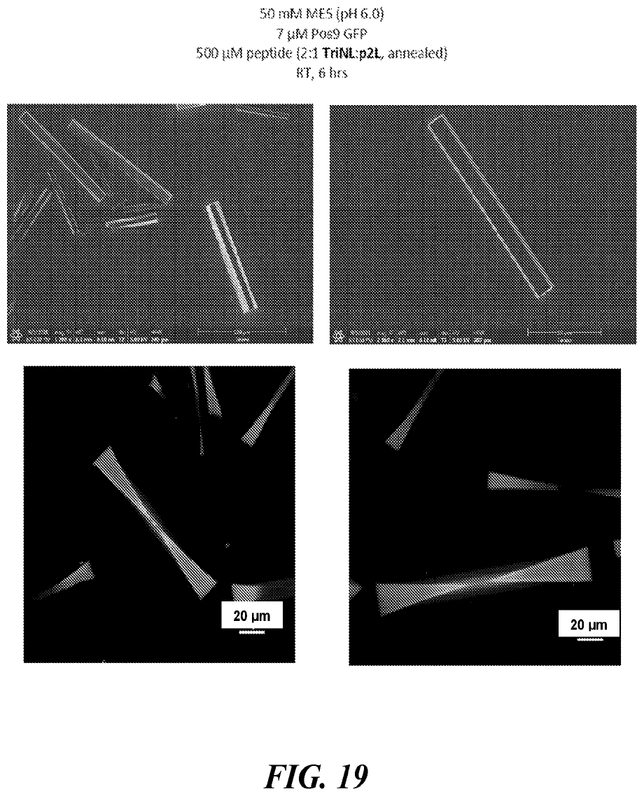

Specifically, at a solution of 2:1 TriNL:p2L was first annealed to form heterotrimers. Once subjected to buffer with protein, large crystals formed with protein incorporation in an hourglass pattern, a characteristic of crystal guest incorporation. The material was collected via centrifugation and imaged via SEM and confocal microscopy. See FIGS. 17-19.

For the purposes of promoting an understanding of the principles presented herein, reference is made to the embodiments illustrated in the drawings and specific language is used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims.

In describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

1. A supramolecular assembly comprising:
a first set of peptide units, each peptide unit of the first set comprising a trimeric coiled-coil peptide comprising a first metal-binding ligand fused to a first end of a trimeric variant of a GCN4 peptide and a second metal-binding ligand fused to a second end of the trimeric variant of a GCN4 peptide, and the first set of peptide units forming a three-dimensional (3D) crystal; and
at least one histidine-tagged (His-tagged) cargo reversibly incorporated into the 3D crystal, each cargo retaining a native structure and functional activity thereof and independently organized within or on the 3D crystal.

2. The supramolecular assembly of claim 1, wherein the 3D crystal undergoes facile dissolution in the presence of a chelator and releases the at least one cargo retaining its native structure and functional activity.

3. The supramolecular assembly of claim 1, further comprising one or more metal ions to promote self-assembly of the 3D crystal, wherein the one or more metal ions are linked to the metal-binding ligands of each peptide unit of the first set, the linkages comprising coordinate covalent bonds, noncovalent bonds, or a combination thereof.

4. The supramolecular assembly of claim 1, wherein the one or more metal ions are divalent metal ions, trivalent metal ions, or a combination of divalent and trivalent metal ions.

5. The supramolecular assembly of claim 3, wherein the one or more metal ions are selected from the group consisting of Ni²⁺, Zn²⁺, Cu²⁺, Co²⁺, Fe²⁺, Co³⁺, Fe³⁺, Rh³⁺, Ru³⁺, and Gd³⁺.

6. The supramolecular assembly of claim 1, wherein:
the first end of the trimeric variant is an N-terminus and the second end of the trimeric variant is a C-terminus;
the first metal-binding ligand comprises nitrilotriacetic acid (NTA); and/or
the second metal-binding ligand comprises di-histidine (His₂).

7. The supramolecular assembly of claim 1, comprising about a 30-70:1 ratio of peptide units of the first set to cargo.

8. (canceled)

9. The supramolecular assembly of claim 1, wherein the at least one cargo comprises a His-tagged, fully folded protein.

10. The supramolecular assembly of claim 1, wherein the at least one cargo comprises a His-tagged fluorescent molecule, a His-tagged oligonucleotide, and/or a His-tagged therapeutic agent.

11-13. (canceled)

14. The supramolecular assembly of claim 1, wherein the at least one cargo reversibly incorporated into the 3D crystal comprises a protein and, when the supramolecular assembly is stored at room temperature, the protein does not undergo substantial denaturation of its functional 3D native structure.

15. The supramolecular assembly of claim 14, wherein at least one cargo is a His-tagged enhanced green fluorescent protein with an N-terminal His₆-tag (EGFP).

16. The supramolecular assembly of claim 15, wherein the EGFP is incorporated into the 3D crystal in an ordered, hourglass pattern.

17. (canceled)

18. The supramolecular assembly of any one of claim 1, further comprising a second set of peptide units, each peptide unit of the second set comprising a trimeric coiled-coil peptide without metal-binding ligands fused thereto, wherein the first and second sets of peptide units in combination form the 3D crystal.

19. The supramolecular assembly of claim 18, wherein each trimeric coiled-coil peptide of the second set has at least 75% sequence identity or more, at least 85% sequence identity or more, at least 90% sequence identity or more, or at least 95% sequence identity or more to SEQ ID NO: 1.

20. (canceled)

21. The supramolecular assembly of claim 18, wherein the trimeric coiled-coil peptide of the second set is encoded by SEQ ID NO: 2 or has at least 75% sequence identity or more, at least 85% sequence identity or more, at least 90% sequence identity or more, or at least 95% sequence identity or more to SEQ ID NO: 2.

22. (canceled)

23. The supramolecular assembly of claim 19, wherein the trimeric variant is encoded by SEQ ID NO: 1 or has at least 75% sequence identity or more, at least 85% sequence identity or more, at least 90% sequence identity or more, or at least 95% sequence identity or more to SEQ ID NO: 1.

24-26. (canceled)

27. A method for the preparation of a supramolecular assembly comprising:
a first set of peptide units, each peptide unit of the first set comprising a trimeric coiled-coil peptide comprising a first metal-binding ligand fused to a first end of a trimeric variant of a GCN4 peptide and a second metal-binding ligand fused to a second end of the trimeric variant of a GCN4 peptide, and the first set of peptide units forming a three-dimensional (3D) crystal; and
at least one histidine-tagged (His-tagged) cargo reversibly incorporated into the 3D crystal, each cargo retaining a native structure and functional activity thereof and independently organized within or on the 3D crystal, the method comprising:
combining a plurality of the first set of peptide units and a plurality of the at least one His-tagged cargo to generate a composition, each peptide unit comprising a trimeric coiled-coil peptide comprising the first metal-binding ligand fused to the first end of a trimeric variant of a GCN4 peptide and the second metal-binding ligand fused to the second end of the trimeric variant of a GCN4 peptide and the composition comprising between about a 30:1 and about a 70:1 ratio of peptide units to His-tagged cargo unit.

28. The method of claim 27, wherein combining further comprises combining a metal source with the plurality of peptide units and the plurality of His-tagged cargo units to generate the composition.

29. The method of claim 27, wherein cargo units comprise a protein, a fluorescent molecule, an oligonucleotide, or a combination of two or more of the foregoing.

30. The method of claim 27, wherein the cargo units comprise a therapeutic agent.

31-35. (canceled)

36. A method for treating a subject experiencing or at risk for experiencing a disease state comprising:
providing a composition comprising a plurality of supramolecular assemblies loaded with cargo, each assembly comprising:
a plurality of peptide units and one or more metal ions to promote self-assembly of the peptide units into a 3D crystal, each peptide unit comprising a trimeric coiled-coil peptide each comprising a NTS ligand fused to an N-terminus of a trimeric variant of a GCN4 peptide and a His₂ ligand fused to a C-terminus of the trimeric variant of a GCN4 peptide, and wherein the one or more metal ions link to the NTS ligand and/or the His₂ ligand of the peptide units to form a 3D crystal, and
a His-tagged cargo reversibly incorporated into the 3D crystal, the cargo exhibiting a functional 3D native structure and independently organized within or on the 3D crystal, wherein the cargo is a therapeutic agent; and
administering the cargo of the supramolecular assemblies to a subject.

37. The method of claim 36, further comprising releasing the cargo from the supramolecular assemblies through facile dissolution prior to administering, wherein the released cargo substantially retains its functional 3D native structure.

38. The method of claim 36, wherein the supramolecular assemblies incorporating the cargo are administered to the subject to affect a prolonged release of the therapeutic agent.

39. The method of claim 37, wherein releasing the cargo comprises applying a chelator to the composition of supramolecular assemblies.

40. The method of claim 36, wherein administering comprises intravenous or subcutaneous injection of the cargo into the subject.

41-48. (canceled)


SEQUENCE LISTING (6 sequences)

SEQ ID NO: 1 - protein, 33 aa, Artificial Sequence
  trimeric coiled-coil peptide variant based on GCN4 leucine zipper sequence
GMKQIEDKIEEILSKIYHIENEIARIKKLIGEG

SEQ ID NO: 2 - protein, 31 aa, Artificial Sequence
  trimeric coiled-coil peptide variant based on GCN4 leucine zipper sequence
MKQIEDKIEEILSKIYHIENEIARIKKLIGE

SEQ ID NO: 3 - protein, 32 aa, Artificial Sequence
  trimeric coiled-coil peptide variant based on a GCN4 leucine zipper sequence
  SITE (3)..(3): Where “X” at position 3 is N, Q, or E
  SITE (18)..(18): Where “X” at position 18 is N, Q, or E
  SITE (25)..(25): Where “X” at position 25 is N, Q, or E
  SITE (28)..(28): Where “X” at position 28 is N, Q, or E
GMXQIEDKIEEILSKIYXIENEIAXIKXLIGE

SEQ ID NO: 4 - protein, 32 aa, Artificial Sequence
  trimeric coiled-coil peptide variant based on a GCN4 leucine zipper sequence
  SITE (7)..(7): Where “X” at position 7 is N, Q, R, or K
  SITE (10)..(10): Where “X” at position 10 is N, Q, R, or K
  SITE (11)..(11): Where “X” at position 11 is N, Q, R, or K
  SITE (32)..(32): Where “X” at position 32 is N, Q, R, or K
GMKQIEXKIXXILSKIYHIENEIARIKKLIGX

SEQ ID NO: 5 - protein, 31 aa, Artificial Sequence
  trimeric coiled-coil peptide variant based on a GCN4 leucine zipper sequence
  SITE (2)..(2): Where “X” at position 2 is N, Q, or E
  SITE (17)..(17): Where “X” at position 17 is N, Q, or E
  SITE (24)..(24): Where “X” at position 24 is N, Q, or E
  SITE (27)..(27): Where “X” at position 27 is N, Q, or E
MXQIEDKIEEILSKIYXIENEIAXIKXLIGE

SEQ ID NO: 6 - protein, 31 aa, Artificial Sequence
  trimeric coiled-coil peptide variant based on a GCN4 leucine zipper sequence
  SITE (6)..(6): Where “X” at position 6 is N, Q, R, or K
  SITE (9)..(9): Where “X” at position 9 is N, Q, R, or K
  SITE (10)..(10): Where “X” at position 10 is N, Q, R, or K
  SITE (31)..(31): Where “X” at position 31 is N, Q, R, or K
MKQIEXKIXXILSKIYHIENEIARIKKLIGX